(19) United States
(12) Patent Application Publication
(10) Pub. No.: US 2026/0047965 A1
(43) Pub. Date: Feb. 19, 2026

(54) WOUND DRESSING

(71) Applicant: ConvaTec Limited, Flintshire (GB)

(72) Inventors: Lucy Ballamy, Llangollen (GB); Sophie Ballamy, Flintshire (GB)

(21) Appl. No.: 19/266,912

(22) Filed: Jul. 11, 2025

Related U.S. Application Data
(63) Continuation of application No. PCT/GB2024/050191, filed on Jan. 24, 2024.
(60) Provisional application No. 63/440,774, filed on Jan. 24, 2023.
(60) Provisional application No. 63/440,787, filed on Jan. 24, 2023.
(60) Provisional application No. 63/440,793, filed on Jan. 24, 2023.

(30) Foreign Application Priority Data
Feb. 27, 2023 (GB) 2302860.8
Feb. 27, 2023 (GB) 2302861.6
Mar. 1, 2023 (GB) 2303030.7

(51) Int. Cl. A61F 13/539; A61F 13/05; A61F 13/15

(52) U.S. Cl. CPC A61F 13/539; A61F 13/05; A61F 13/15707; A61F 2013/53958

(57) ABSTRACT

A wound dressing (10) having a periphery having an outline. The wound dressing comprises an absorbent pad having an outline. The outline of the periphery and the outline of the absorbent pad have the same shape. The shape comprising two lobes, the boundary between the two lobes defining a flex line, and at least one lobe comprising a least one notch.

### TECHNICAL FIELD OF THE INVENTION

The present invention relates to the shape of a wound dressing.

### BACKGROUND TO THE INVENTION

It is known to make wound dressings for use on wounds to aid healing and prevent infections. Such dressings may be required to adhere to a patient's body in whichever location the wound is present. This may require a dressing to be adhered to areas which are variably or highly contoured or which experience a relatively high level of movement. This can make the initial adherence of the dressing challenging and limit subsequent wear time.

To address this problem, practitioners will often use additional securing means to attach a dressing which results in a additional time, cost and reduced comfort for the patient. To help reduce this, manufacturers efforts have often focussed on providing dressings with site specific shapes. However, this not only complicates design and manufacturing of the different shape types and sizes of the dressings, but also requires health care providers to stock many different types and sizes of dressings. Further, it requires practitioners to be educated and/or experienced in many different types of wound dressing to reduce the risk of the dressings being used incorrectly.

The present invention seeks to provide a wound dressing having a shape which helps alleviate some of the issues of existing dressing shapes.

### SUMMARY OF THE INVENTION

The present invention provides a wound dressing according to the appended claims.

In broad terms, the invention provides a wound dressing. The wound dressing may have a periphery. The periphery may have an outline. The wound dressing may comprise an absorbent pad. The absorbent pad may have an outline. The outline of the periphery and the outline of the absorbent pad may have the same shape. The shape of the outline of the periphery of the absorbent pad and/or the periphery of the wound may comprise two lobes. The boundary between the two lobes may define a flex line. At least one lobe may comprise at least one notch.

According to a first aspect of the invention there is provided a wound dressing having a periphery having an outline, the wound dressing comprising an absorbent pad having an outline, wherein the outline of the periphery and the outline of the absorbent pad have the same shape, the shape comprising two lobes, the boundary between the two lobes defining a flex line, at least one lobe comprising at least one notch.

The outline of the absorbent pad and outline of the periphery of the dressing having the same shape means they are geometrically similar; the relative arrangement and proportions of the two outlines are the same, but they may have different sizes, for example the wound dressing periphery may be uniformly scaled up with respect to the absorbent pad periphery.

The one, or more, or each notch may be provided on the periphery of the wound dressing. The one, or more, or each notch may be provided on the absorbent pad. The one, or more, or each notch may be provided on a periphery of the absorbent pad. Advantageously, by providing a wound dressing wherein the absorbent pad and periphery of the dressing have a shape comprising two lobes the wound dressing exhibits improved conformity to contoured wound sites, improving the fit on said sites thereby promoting improved wound healing. Likewise the provision at least one notch in at least one lobe further improves the ability of the dressing to conform to contoured wound sites as it provides a point at which a fold can be preferentially formed and additionally allows the dressing to be formed into a shallow cone shape. The advantages are synergistic, the combination of the features provides a superior ability for the dressing to conform to the wound site.

The one, or more, or each notch may extend radially inwards from the periphery of the wound dressing. The one, or more, or each notch may extend radially inwards from the periphery of the absorbent pad.

The wound dressing may comprise a wound facing side and an opposing second side. The notch may extend through the wound dressing, that is the notch may extend from the wound facing side to the opposing second side.

The absorbent pad may comprise a wound facing side and an opposing second side. The notch may extend through the absorbent pad, that is the notch may extend from the wound facing side to the opposing second side.

A line bisecting the notch may be at an acute angle to the flex line. Where present the bisecting line divides the notch in to two equal sections. The notch may be symmetric about the bisecting line.

Accordingly, in a preferred embodiment of the invention there is provided a wound dressing having a periphery having an outline, the wound dressing comprising an absorbent pad having an outline, wherein the outline of the periphery and the outline of the absorbent pad have the same shape, the shape comprising two lobes, the boundary between the two lobes defining a flex line, at least one lobe comprising at least one notch, wherein a line bisecting the notch is at an acute angle to the flex line. Each notch may be bisected by a line. Each bisecting line may be at an acute angle to the flex line.

The acute angle may be less than 90 degrees. The acute angle may be between 1 and 89 degrees, preferably between 30 and 60 degrees, more preferably between 40 and 50 degrees, for example 45 degrees.

The at least one notch may comprise two straight edges. The two straight edges may be arranged at 90 degrees to one another. The two straight edges may be arranged at less than 90 degrees to one another. The straight edges of the notch may have a length of between 4 and 12 mm, preferably between 6 and 10 mm, more preferably between 7 and 9 mm, for example 8 mm.

The at least one notch may comprise two curved edges. The two curved edges may intersect at an angle of 90 degrees. The two curved edges may intersect at an angle of less than 90 degrees.

The wound dressing may comprise at least two layers. The two layers may be the absorbent pad and a wound-site adhesive layer. The wound-site adhesive layer may be for adhering the wound dressing to the wound. The wound-site adhesive layer may have at least two lobes. The wound-site adhesive layer may have two lobes. The wound site adhesive layer may have the same shape as the absorbent pad. The periphery of the wound-site adhesive layer may define the periphery of the wound dressing. The wound-site adhesive layer may extend beyond the outline of the absorbent pad. The radial extent to which the wound-site adhesive layer extends beyond the outline of the absorbent pad may remain constant. The radial extent to which the wound-site adhesive layer extends beyond the outline of the absorbent pad may remain constant around the whole periphery of the absorbent pad. The radial extent to which the wound-site adhesive layer extends beyond the outline of the absorbent pad may remain constant other than in the region of the at least one notch. The wound-site adhesive layer and absorbent pad may radially overlap such that the wound-site adhesive layer is adhered to the absorbent pad. The radial overlap between the absorbent pad and wound-site adhesive layer may be uniform around the full perimeter of the overlap. A radially outer edge of the wound-site adhesive layer may be coterminous with a radially outer edge of a backing layer. A radially inner edge of the wound-site adhesive layer may correspond to a radially outer peripheral edge of the absorbent pad.

A uniform radial overlap of the wound-site adhesive layer about the entire perimeter is advantageous as it ensures the dressing remains adhered to the wound-site even when the dressing is deformed to fit the wound site.

A notch may be provided on the absorbent pad. A notch may be provided on the wound-site adhesive layer. At least one notch may be provided on the absorbent pad. At least one notch may be provided on the wound-site adhesive layer. A notch may be provided on both the absorbent pad and the wound-site adhesive layer. At least one notch may be provided on both the absorbent pad and the wound-site adhesive layer. At least two notches may be provided on the absorbent pad. At least two notches may be provided on the wound-site adhesive layer. A notch may be provided on both the absorbent pad and the wound-site adhesive layer. At least one notch may be provided on both the absorbent pad and the wound-site adhesive layer. At least two notches may be provided on each of the absorbent pad and the wound-site adhesive layer. Four notches may be provided on the absorbent pad. Four notches may be provided on the wound-site adhesive layer. Four notches may be provided on each of the absorbent pad and the wound-site adhesive layer. One, or more, or each notch on the absorbent pad may be aligned with a notch on the wound-site adhesive layer. One, or more, or each notch on the absorbent pad may be aligned with the notch on the wound-site adhesive layer such that a single line bisects both notches. A plurality of notches may be provided on both the absorbent pad and the wound-site adhesive layer. One, or more, or each notch on the absorbent pad may be aligned with a notch on the wound-site adhesive layer to form a notch pair. Both notches in a notch pair may have the same profile. The notches in a notch pair may have different profiles. One, or more, or each notch pair may have the same profile. One, or more, or each notch pair may have a straight edged profile. The angle between the straight edges of the notch may be 90 degrees. Both notches in a notch pair may have the same bisecting line.

By providing notches on both the absorbent pad and the adhesive layer, the dressing can be folded to conform to the wound site without the adhesive layer rippling or bulging such that the adhesive is not in contact with the wound site. This ensures that the dressing is secured to the wound site and reduces the risk of infection where an incomplete seal of the wound is provided by the dressing.

The one, or more, or each notch may be a V-shaped notch. The one, or more, or each notch may have a curved profile. The one, or more, or each notch may be semi-circular. The one, or more, or each notch may be a curved V-shape.

The one, or more, or each absorbent pad notch may be a V-shaped notch. The one, or more, or each absorbent pad notch may have a curved profile. The one, or more, or each absorbent pad notch may be semi-circular. The one, or more, or each absorbent pad notch may be a curved V-shape.

The one, or more, or each wound-site adhesive layer notch may be a V-shaped notch. The one, or more, or each wound-site adhesive layer notch may have a curved profile. The one, or more, or each wound-site adhesive layer notch may be semi-circular. The one, or more, or each wound-site adhesive layer notch may be a curved V-shape.

The wound dressing may comprise a wound contact layer. The wound dressing may comprise a transmission layer. The wound dressing may comprise a fluid distribution layer. The wound dressing may comprise a wicking layer. The wound dressing may comprise an absorbency layer. The wound dressing may comprise a superabsorbency layer. The wound dressing may comprise a foam layer. The wound dressing may comprise a pressure distribution layer. The wound dressing may comprise a gauze layer. The wound dressing may comprise an intralayer adhesive layer.

Where the wound dressing comprises an absorbent pad and a wound-site adhesive layer, the wound dressing may further comprise a further layer selected from the following: a wound contacting layer, a covering layer, a fluid distribution layer, a wicking layer, an absorbency layer, a superabsorbency layer, a transmission layer, a foam layer, a pressure distribution layer, a gauze layer and/or an intralayer adhesive layer. The further layer may have the same outline as the absorbent pad. The further layer may have the same shape as the absorbent pad. The further layer may have the same outline as the wound-stie adhesive layer. The further layer may have the same shape as the wound-stie adhesive layer. The further layer may comprise one or more notches. One or more or each notch in the further layer may align with a notch on the absorbent pad.

Where the wound dressing comprises an absorbent pad and a wound-site adhesive layer, the wound dressing may further comprise two further layers selected from the following: a wound contacting layer, a covering layer, a fluid distribution layer, a wicking layer, an absorbency layer, a superabsorbency layer, a transmission layer, a foam layer, a pressure distribution layer, a gauze layer and/or an intralayer adhesive layer. One or each of the further layers may have the same outline as the absorbent pad. One or each of the further layers may have the same shape as the absorbent pad. One or each of the further layers may have the same outline as the wound-stie adhesive layer. One or each of the further layers may have the same shape as the wound-stie adhesive layer. One or each of the further layers may comprise one or more notches. One or more or each notch in the further layer(s) may align with a notch on the absorbent pad.

Where the wound dressing comprises an absorbent pad and a wound-site adhesive layer, the wound dressing may further comprise three or more further layers (for example four layer, or five layer, or six layers) selected from the following: a wound contacting layer, a covering layer, a fluid distribution layer, a wicking layer, an absorbency layer, a superabsorbency layer, a transmission layer, a foam layer, a pressure distribution layer, a gauze layer and/or an intralayer adhesive layer. One or more or each of the further layers may have the same outline as the absorbent pad. One or more or each of the further layers may have the same shape as the absorbent pad. One or more or each of the further layers may have the same outline as the wound-stie adhesive layer. One or more or each of the further layers may have the same shape as the wound-stie adhesive layer. One or more or each of the further layers may comprise one or more notches. One or more or each notch in the further layer(s) may align with a notch on the absorbent pad.

A lobe may have a part-circular outline. Each lobe may have a part-circular outline. Each lobe may be of equal size. The shape of the wound dressing may comprise two overlapping circles. The lobes may overlap such that each lobe comprises more than half a circle. Each lobe may overlap by between 10-40% of their perimeter, preferably between 20 and 30%, more preferably between 22 and 28%, for example 25%.

Each lobe may have a centre point. The centre point may be the origin about which the (part) circular outline of the lobe is defined. Where the wound dressing has two lobes, the centre points of two lobes may have a separation of between 50 and 90 mm, preferably between 60 and 80 mm, more preferably between 65 and 75 mm, for example 70 mm.

One or more or each lobe of the wound-site adhesive layer may have a radius of between 50 and 100 mm, preferably between 60 and 90 mm, more preferably between 70 and 80 mm for example 75 mm.

One or more or each lobe of the absorbent pad may have a radius of between 25 and 75 mm, preferably between 40 and 60 mm, more preferably between 45 and 55 mm for example 50 mm.

A region of the wound-site adhesive layer between the peripheral edge thereof and the peripheral edge of the absorbed pad may define a border region. The border region may have a radial width of between 15 and 35 mm, preferably between 20 and 30 mm, more preferably between 23 and 27 mm, for example 25 mm.

The wound dressing may have a (first) line of symmetry defined by the flex line. By providing the wound dressing symmetrical about the flex line the dressing may be more easily applied, being less dependent upon orientation. The wound dressing may have a second line of symmetry. The second line of symmetry may be arranged perpendicular to, and at the mid-point of the flex line. Providing a wound dressing with two degrees of symmetry further improves the ease of application as the dressing is even less dependent upon orientation.

Where the flex line meets the periphery, the periphery may have an inflection point(s). The inflection point(s) may be formed by the convergences of the peripheries of the two lobes. The internal angle of the inflection point(s) may be greater than 180 degrees.

Where the flex line meets the periphery of the absorbent pad, the periphery may have pad inflection point(s). The pad inflection point(s) may be formed by the convergence of the peripheries of the two lobes of the absorbent pad. The internal angle of the pad inflection point(s) may be greater than 180 degrees.

Where the flex line meets the periphery of the wound-site adhesive layer, the periphery may have inflection point(s). The wound-site adhesive layer inflection point(s) may be formed by the convergence of the peripheries of the two lobes of the wound-site adhesive layer. The internal angle of the wound-site absorbent pad inflection point(s) may be greater than 180 degrees.

The width of the wound dressing may be measured parallel to the flex line. The width of the wound dressing may be a minima at the flex line. The width of the wound dressing at the flex line may be less than the width at a maxima. The maxima may be measured at a point crossing the centre of a lobe. The width of the wound dressing at the flex line may between 70 and 95% the maximum width, preferably between 80 and 95%, more preferably between 85 and 91%, for example 88%. The width of the wound dressing at the flex line may be between 100 and 180 mm, preferably between 110 and 150 mm, more preferably between 125 and 140 mm, for example 132 mm. The wound dressing may have a maximum width of between 100 and 200 mm, preferably the maximum width is between 125 and 175 mm, more preferably between 140 and 160 mm, for example 150 mm.

The length of the wound dressing may be measured perpendicular to the flex line. The wound dressing may have a length of between 150 and 300 mm, preferably the length is between 200 and 240 mm, more preferably between 210 and 230 mm, for example 220 mm.

The width of the absorbent pad may be measured parallel to the flex line. The width of the absorbent pad may be a minima at the flex line. The width of the absorbent pad at the flex line may be less than the width at a maxima. The maxima may be measured at a point crossing the centre of a lobe. The width of the absorbent pad at the flex line may between 60 and 80% the maximum width, preferably between 65 and 75%, more preferably between 69 and 73%, for example 71%. The width of the absorbent pad at the flex line may be between 60 and 80 mm, preferably between 65 and 75 mm, more preferably between 69 and 73 mm, for example 71 mm. The absorbent pad may have a maximum width of between 50 and 150 mm, preferably the maximum width is between 75 and 125 mm, more preferably between 90 and 110 mm, for example 100 mm.

The length of the absorbent pad may be measured perpendicular to the flex line. The absorbent pad may have a length of between 150 and 300 mm, preferably the length is between 200 and 240 mm, more preferably between 210 and 230 mm, for example 220 mm.

The line(s) bisecting the one or more or each notch may intersect the flex line. The line(s) bisecting the one or more or each notch may intersect the flex line at the edge of the absorbent pad. The line(s) bisecting the one or more or each notch may intersect the flex line at the inflection point. The line(s) bisecting the one or more or each notch pair may intersect the flex line. The line(s) bisecting the one or more or each notch pair may intersect the flex line at the edge of the absorbent pad. The line(s) bisecting the one or more or each notch pair may intersect the flex line at the inflection point.

The arrangement whereby the one, or more, or each notch bisecting line(s) intersect with the flex line at an inflection point, also aids the folding of the dressings about the bisecting lines, and in particular means that the folding of one lobe along a notch flex line does not affect the shape of the other lobe (since the fold line does not extend into the other lobe).

Perforations may be provided in the wound-site adhesive layer. The perforations may be provided along the flex line. The perforations may be provided from a periphery of the wound-site adhesive layer to a periphery of the absorbent pad. The perforations may be provided between a periphery of the wound-site adhesive layer and a periphery of the absorbent pad. The perforations may be provided from a periphery of the wound-site adhesive layer to between 40 and 60% of the distance to a periphery of the absorbent pad. Preferably the perforations are provided between 45 and 55% of the distance between the peripheries of the wound-site adhesive layer and absorbent pad, more preferably 50%.

The perforations may extend between 5 and 25 mm from the peripheral edge of the wound-site adhesive layer, preferably between 10 and 20 mm more preferably between 13 and 17 mm, for example 15 mm.

The perforations provide for an improved seal when the wound dressing is folded about the flex line as they allow the adhesive layer to be layered upon itself at the flex line.

At least one lobe may comprise at least two notches. Each lobe may comprise at least two notches. Lines bisecting the respective of the at least two notches in the at least one lobe may intersect at a point other than on the flex line.

Accordingly, in a preferred embodiment of the invention there is provided a wound dressing having a periphery having an outline, the wound dressing comprising an absorbent pad having an outline, wherein the outline of the periphery and the outline of the absorbent pad have the same shape, the shape comprising two lobes, the boundary between the two lobes defining a flex line, at least one lobe comprising at least two notches, wherein lines bisecting the at least two notches intersect at a point other than on the flex line.

The lines bisecting the at least two notches may intersect in their corresponding lobe. The lines bisecting the at least two notches may intersect at the centre of the corresponding lobe. The lines bisecting the at least two notches may intersect at the centre of the circle defining the lobe.

One or more or each lobe may be provided with at least two notch pairs. At least two of the notch pairs may intersect in their corresponding lobe. The lines bisecting at least two notch pairs may intersect at the centre of their corresponding lobe. The lines bisecting at least two notch pairs may intersect at the centre of the circle defining the lobe.

The wound dressing may have two lobes. Each of the two lobes may have one notch. Each of the two lobes may have at least one notch. Each of the two lobes may comprise at least two notches. Each of the two lobes may have two notches. Lines bisecting the two notches on each corresponding lobe may intersect at a point other than on the flex line. The bisecting lines of the notches on both lobes may intersect at the same point. The bisecting lines of the notches on both lobes may intersect at different points. The lines may intersect in their respective lobes. The lines may intersect at the centre of their respective lobe. The lines may intersect at the centre of the circle defining each lobe.

Advantageously, where the bisecting lines intersect within their respective lobes, the conforming of each lobe can be independent of the other as the fold lines do not extend into the other lobe, this means that more complexly contoured wound sites can be treated using the dressing.

The wound dressing may be provided in packaging. The packaging may provide a sterile cavity in which the wound dressing is provided.

The wound dressing may be configured for use in a pressure gradient wound therapy system. The wound dressing may comprise a covering layer. An adhesive may be applied to the covering layer. Accordingly, the covering layer may be the wound-site adhesive layer. The covering layer may comprise an aperture. The aperture may be arranged off centre of the wound dressing. The aperture may be arranged in one of the lobes of the wound dressing. The covering layer may comprise a port. The port may be arranged over the aperture. The port may be arranged around the port.

The wound dressing may be provided in a kit for use in a pressure gradient wound therapy system. The kit may further comprise at least one sealing stip. The sealing strip may comprise an adhesive backed tape. The sealing strip may be provided with a release layer. The release layer may comprise a plurality of release strips. The sealing strip may be shaped to overlap at least a portion of the perimeter of the wound dressing. The kit may comprise two sealing strips. Each of the two sealing strips may have the same shape. Each of the two sealing strips may both cover one half of each lobe. Each of the two sealing strips may extend about the perimeter of the wound dressing from a midpoint between the two notches on a first lobe to the midpoint between the two notches on a second lobe. The kit may comprise four sealing strips. Each of the four sealing strips may have the same shape. Each of the four sealing strips may cover one half of one lobe. Each of the four sealing strips may extend about the perimeter of the wound dressing from a midpoint between the two notches on a lobe and an inflection point between the two lobes. The kit may comprise six sealing strips. Each of the six sealing strips may have the same shape. The sealings strips may be provided with a release tab.

According to a second aspect, the present disclosure may provide a method of applying a dressing comprising: locating a dressing as disclosed or claimed herein over a wound site having a variously contoured surface; aligning the flex line and/or one or more notch bisecting lines with one or more ridges on the contoured surface; adhering a first lobe to the wound; folding the wound dressing about the one or more ridges; adhering the second lobe.

In a further aspect, the present disclosure provides a wound dressing comprising at least one layer having a tear-drop shaped outline. The tear-drop shape may comprise a semi-circular portion and a tapered portion. The tapered portion may terminate in a rounded edge.

According to a third aspect of the invention there is provided a wound dressing comprising at least one layer having a tear-drop shaped outline, the tear-drop shape comprising a semi-circular portion and a tapered portion, wherein the tapered portion terminates in a rounded edge.

A wound dressing comprising a layer with a tear-drop shape provides an improved conformity on variably or highly contoured wound sites. These areas may include a shoulder, armpit, sacral region, breast, hip and heel, for example.

The tapered potion may be curved. The tapered portion may be convexly curved. The periphery of the circular portion and the periphery of the tapered portion may meet at two interface points. The tapered portion will (by definition) have a wider end and a narrower end. The interface points may be at the wider end of the tapered portion. The rounded edge may be at the narrower end of the tapered portion.

The wound dressing may comprise a major axis aligned with, for example co-axial with the taper of the tapered portion. The wound dressing may comprise a major axis extending along the length of the dressing. The wound dressing may be symmetrical about the major axis. An apex of the tapered portion may be arranged on the major axis. An apex of the semi-circular portion may be arranged on the major axis. The wound dressing may have a transverse axis arranged perpendicular to the major axis. The transverse axis may extend between the two interface points of the wound dressing perimeter. The width of the dressing may be defined as aligned with, for example co-axial with the transverse axis. The major axis and transverse axis may intersect at a centre point of the semi-circular portion (the centre point being defined as the origin of a circle defined by the semi-circular portion). A central region may be defined about the centre point of the semi-circular portion. The central region may be defined as a circular region about the centre point having a radius less than the radius of the semi-circular portion. The central region may be defined as a circular region about the centre point having a radius equal to the radius of the semi-circular portion.

The width of the dressing may be at a maxima at the point between the two inflection points. The transverse axis may define the boundary between the semi-circular portion and the tapered portion.

The length of the dressing may be defined as aligned with, for example co-axial with the major axis. The length of the taper may be defined as the distance along the major axis from the point of intercept with the transverse axis to the rounded edge at the apex of the taper. The length of the taper may be equal to the width of the dressing. The length of the taper may be greater than the width of the dressing.

The wound dressing may comprise a wound contact layer. The wound dressing may comprise a covering layer. The wound dressing may comprise a wound-site adhesive layer. The wound dressing may comprise an absorbent layer. The wound dressing may comprise a transmission layer. The wound dressing may comprise a fluid distribution layer. The wound dressing may comprise a wicking layer. The wound dressing may comprise an absorbency layer. The wound dressing may comprise a superabsorbency layer. The wound dressing may comprise a foam layer. The wound dressing may comprise a pressure distribution layer. The wound dressing may comprise a gauze layer. The wound dressing may comprise an intralayer adhesive layer.

A/the wound contact layer may have a tear-drop shaped outline. A/the covering layer may have a tear-drop shaped outline. A/the wound-site adhesive layer may have a tear-drop shaped outline. An/the absorbent layer may have a tear-drop shaped outline. A/the transmission layer may have a tear-drop shaped outline. A/the fluid distribution layer may have a tear-drop shaped outline. A/the wicking layer may have a tear-drop shaped outline. A/the absorbency layer may have a tear-drop shaped outline. A/the superabsorbency layer may have a tear-drop shaped outline. A/the foam layer may have a tear-drop shaped outline. A/the pressure distribution layer may have a tear-drop shaped outline. A/the gauze layer may have a tear-drop shaped outline. A/the intralayer adhesive layer may have a tear-drop shaped outline. The wound-site adhesive layer may be arranged on the covering layer and have the same outline. The wound dressing (i.e. the outer periphery of the dressing as a whole) may have a tear-drop shaped outline.

One or more layers of the wound dressing may have an ovate (i.e. egg-shaped) outline.

The wound dressing (i.e. the outer periphery of the dressing as a whole) may have an ovate outline. A wound contact layer may have the ovate outline. A covering layer may have the ovate outline. A wound-site adhesive layer may have the ovate outline. An absorbent layer may have the ovate outline. A transmission layer may have the ovate outline. A/the fluid distribution layer may have an ovate outline. A/the wicking layer may have an ovate outline. A/the absorbency layer may have an ovate outline. A/the superabsorbency layer may have an ovate outline. A/the foam layer may have an ovate outline. A/the pressure distribution layer may have an ovate outline. A/the gauze layer may have an ovate outline. A/the intralayer adhesive layer may have an ovate outline. The wound-site adhesive layer may be arranged on the covering layer and have the same outline.

The ovate outline may be larger than the teardrop shaped outline. The ovate outline and the teardrop shaped outline may be concentric.

The ovate outline may have a semi-circular portion and a tapered portion. The tapered potion may be curved. The tapered portion may be convexly curved. The periphery of the circular portion and the periphery of the tapered portion may meet at two interface points. The tapered portion will (by definition) have a wider end and a narrower end. The interface points may be at the wider end of the tapered portion. The rounded edge may be at the narrower end of the tapered portion.

The rounded edge of the tapered portion of the teardrop shaped outline may be a rounded point, having a substantially smaller radius than the semi-circular portion, for example less than 40%, less than 30%, less than 20%, of the radius of the semi-circular portion.

The rounded edge of the tapered portion of the ovate outline may be less pointy, in relative terms, than the rounded edge of the tapered portion of the teardrop shaped outline. For example, the rounded edge of the tapered portion of the ovate outline may have a radius of less than 70%, less than 60%, less than 50% or less than 45% of the semi-circular portion. The rounded edge of the tapered portion of the ovate outline may have a radius of at least 5%, at least 10%, at least 20% or at least 25% of the radius of the semi-circular portion of the ovate outline.

The round edge of the tapered portion of the teardrop shaped outline may have a radius of less than 50%, less than 40% or less than 30% of the radius of the rounded edge of the tapered portion of the ovate outline. The round edge of the tapered portion of the teardrop shaped outline may have a radius of more than 5%, more than 10%, more than 20% or more than 35% of the radius of the rounded edge of the tapered portion of the ovate outline.

The central region may be defined as a circle about the centre point having the same radius as the semi-circular portion of the wound contact layer.

The wound dressing may comprise at least two layers. The wound dressing may comprise a wound contact layer and a covering layer. The covering layer may extend beyond a perimeter of the wound contact layer. The covering layer may extend an equal distance beyond the perimeter of the wound contact layer about the entire perimeter. An adhesive may be provided on the border of the covering layer. An adhesive may be provided on the portion of the covering layer which extends beyond the perimeter of the wound contact layer. A region of the covering layer extending between a perimeter of the wound contact layer and a perimeter of the covering layer may be provided with an adhesive. The wound dressing may comprise a wound contacting layer and a wound-site adhesive layer for adhering the wound dressing to the wound site. The wound-site adhesive layer may extend beyond a perimeter of the wound contact layer. The wound-site adhesive layer may extend an equal distance beyond the perimeter of the wound contact layer about the entire perimeter. An adhesive may be provided on the border of the wound-site adhesive layer. An adhesive may be provided on the portion of the wound-site adhesive layer which extends beyond the perimeter of the wound contact layer.

The wound dressing may comprise at least two layers selected from the following: a wound contacting layer, a wound-site adhesive layer, a covering layer, a fluid distribution layer, a wicking layer, an absorbency layer, a superabsorbency layer, a transmission layer, a foam layer, a pressure distribution layer, a gauze layer and/or an intralayer adhesive layer.

The wound dressing may comprise at least three layers selected from the following: a wound contacting layer, a wound-site adhesive layer, a covering layer, a fluid distribution layer, a wicking layer, an absorbency layer, a superabsorbency layer, a transmission layer, a foam layer, a pressure distribution layer, a gauze layer and/or an intralayer adhesive layer. Preferably, the wound dressing may comprise a wound contacting layer, a covering layer and one further layer listed above. More preferably the further layer is an absorbency layer. The further layer may be arranged between the wound contacting layer and the covering layer. The further layer may have the same outline as the wound contact layer. The further layer may have a tear drop shaped outline. The further layer may have an ovate outline. The wound contact layer and the further layer may be the same size. The wound contact layer and the further layer may be different sizes. The further layer may the larger than the wound contact layer. The further layer may be smaller than the wound contact layer. The covering layer may be the same shape as the wound contact layer and/or further layer. The covering layer may be larger than the wound contact layer and/or further layer.

The wound dressing may comprise at least four layers selected from the following: a wound contacting layer, a wound-site adhesive layer, a covering layer, a fluid distribution layer, a wicking layer, an absorbency layer, a superabsorbency layer, a transmission layer, a foam layer, a pressure distribution layer, a gauze layer and/or an intralayer adhesive layer. Preferably, the wound dressing may comprise a wound contacting layer, a covering layer and two further layers listed above. More preferably, the two further layers are an absorbency layer and a pressure distribution layer. The further layers may be arranged between the wound contacting layer and the covering layer. Where the further layers are an absorbency layer and a pressure distribution layer, the layers may be ordered: the wound contact layer, the absorbency layer, the pressure distribution layer and the covering layer. One or both further layers may have the same outline as the wound contact layer. The two further layers may have the same outline. The two further layers may have a different outline. One or both further layers may have a tear drop shaped outline. One or both further layers may have an ovate outline. The wound contact layer and one or both further layers may be the same size. The wound contact layer and one or both further layers may be different sizes. One or both further layers may the larger than the wound contact layer. One or both further layers may be smaller than the wound contact layer. The covering layer may be the same shape as the wound contact layer and/or one or both further layers. The covering layer may be larger than the wound contact layer and/or one or both further layers. Where the further layers are an absorbency layer and a pressure distribution layer, the wound contact layer, absorbency layer and pressure distribution layer may have the same tear-drop shape and the covering layer may be an ovate shape.

The wound dressing may comprise at least five layers selected from the following: a wound contacting layer, a wound-site adhesive layer, a covering layer, a fluid distribution layer, a wicking layer, an absorbency layer, a superabsorbency layer, a transmission layer, a foam layer, a pressure distribution layer, a gauze layer or an intralayer adhesive layer. Preferably, the wound dressing may comprise a wound contacting layer, a covering layer and three further layers listed above. More preferably the three further layers may be an absorbency layer, a pressure distribution layer and a wound-site adhesive layer. The further layers may be arranged between the wound contacting layer and the covering layer. Where the further layers are an absorbency layer and a pressure distribution layer and wound-site adhesive layer, the layers may be ordered: the wound contact layer, the absorbency layer, the pressure distribution layer, wound-site contact layer and the covering layer. One or more or each further layer may have the same outline as the wound contact layer. Two of the three further layers may have the same outline. The three further layers may have the same outline. The three further layers may have a different outline. One or more or each further layer may have a tear drop shaped outline. One or more or each further layer may have an ovate outline. The wound contact layer and one or more or each further layer may be the same size. The wound contact layer and one or more or each further layer may be different sizes. One or more or each further layer may the larger than the wound contact layer. One or more or each further layer may be smaller than the wound contact layer. The covering layer may be the same shape as the wound contact layer and/or one or more or each further layer. The covering layer may be larger than the wound contact layer and/or one or more or each further layer. Where the further layers are an absorbency layer, a pressure distribution layer and a wound-site adhesive layer; the wound contact layer, absorbency layer and pressure distribution layer may have the same tear-drop shape and the covering layer and wound-site adhesive layer may be an ovate shape.

A region of the covering layer within that defined by an/the adhesive border may be closed, seal and/or uninterrupted. This restricts and or prevents microbes, bacteria and the like from entering the wound site.

The wound dressing may comprise a release layer, the release layer being removable to reveal the adhesive border.

The radial extent to which the wound-site adhesive layer extends beyond the outline of the absorbent pad may remain constant. The wound-site adhesive layer and wound contact layer may radially overlap such that the wound-site adhesive layer is adhered to the wound contact layer. The radial overlap between the wound contact layer and wound-site adhesive layer may be uniform around full circumference of the overlap. A radially outer edge of the wound-site adhesive layer may be coterminous with a radially outer edge of the covering layer. A radially inner edge of the wound-site adhesive layer may correspond to a radially outer peripheral edge of the wound contact layer.

An outline of the one or more or each layer(s) may have the same shape. The outline of the one or more or each layer(s) may have the same shape as the outline of the wound dressing.

The outline of the layer(s) and outline of the dressing having the same shape means they are geometrically similar, the relative arrangement and proportions of the two outlines are the same but they may have different sizes, for example the wound dressing outline may be uniformly scaled up with respect to the absorbent pad outline.

In a preferred embodiment, (a) the wound site adhesive layer and/or the covering layer have an ovate outline, and (b) the wound contact layer, the absorbent layer and/or the transmission layer have a teardrop shaped outline.

The wound dressing may have a length of between 100 and 300 mm, preferably between 200 and 300 mm, more preferably between 225 and 375 mm, for example 270 mm. The wound dressing may have a width of between 100 and 250 mm, preferably between 150 and 200 mm, more preferably between 165 and 175, for example 190 mm. The semi-circular portion may have a radius of between 50 and 100 mm, preferably between 70 and 90 mm, more preferably between 80 and 90 mm for example 95 mm. The taper may have a length of between 125 and 200 mm, preferably between 150 and 180 mm, more preferably between 160 and 170 mm, for example 175 mm. The rounded edge of the tapered portion may have a radius of between 20 and 50 mm, preferably between 25 and 45 mm, more preferably between 30 and 40 mm, for example 35 mm.

The wound contact layer may have a length of between 100 and 300 mm, preferably between 150 and 250 mm, more preferably between 175 and 225 mm, for example 200 mm. The wound contact layer may have a width of between 80 and 160 mm, preferably between 100 and 140 mm, more preferably between 110 and 130, for example 120 mm. The semi-circular portion may have a radius of between 40 and 800 mm, preferably between 50 and 70 mm, more preferably between 55 and 65 mm for example 60 mm. The taper may have a length of between 100 and 180 mm, preferably between 120 and 150 mm, more preferably between 135 and 145 mm, for example 140 mm. The rounded edge of the tapered portion may have a radius of between 1 and 9 mm, preferably between 5 and 15 mm, more preferably between 8 and 12 mm, for example 10 mm.

The border region may have a thickness of between 10 and 50 mm, preferably between 20 and 40 mm, more preferably between 30 and 40 mm for example 35 mm.

The wound dressing may have a thickness between 1 mm to 20 mm, or 2 mm to 10 mm, or 3 mm to 7 mm, for example.

The wound dressing may be configured for use with a source of non-atmospheric pressure. The wound dressing may be configured for use in a pressure gradient wound therapy system. The wound dressing may be configured for use in negative pressure wound therapy. The wound dressing may be configured for use in positive pressure wound therapy.

When used herein and throughout the specification the term “pressure gradient wound therapy system” is intended to cover a wound therapy system wherein a pressure differential (either positive or negative) is applied between a sealed region of the wound dressing and the surrounding environment.

As used herein, negative pressure wound therapy is a therapeutic technique using a suction dressing to remove excess exudation and promote healing in acute or chronic wounds. A vacuum of −50 to −200 mm Hg, or −75 to −150 mm Hg may be applied with typical negative pressure of −80 to −130 mm Hg, −100 to −130 mm Hg, or often about −125 mm Hg being applied to a wound.

For positive pressure wound therapy, a net positive pressure is applied to the wound, which may include providing simultaneous aspiration and irrigation of the wound. Positive pressure wound therapy may be carried out at a positive pressure of up to 50% atm., typically at a low positive pressure of up to 20% atm., more usually up to 10% atm. at the wound. Positive pressure wound therapy is known and referred to in US20180140755.

The wound dressing may further comprise a wound contact layer having a first surface for contacting a wound and an opposing second surface. The wound dressing may further comprise a covering layer. The covering layer may have a first surface facing the wound contact layer. The first surface may define a wound dressing cavity. The covering layer may have a second surface opposing the first surface. The wound contact layer may be arranged within the cavity. The covering layer may further comprise an aperture configured to provide fluid communication between the wound dressing cavity and a source of non-atmospheric pressure. The covering layer may further comprise an aperture configured to provide fluid communication between the wound dressing cavity and a pressure gradient source.

As such, the present disclosure provides for a wound dressing comprising at least one layer having a tear-drop shaped outline, wherein the wound dressing is configured for use with a source of non-atmospheric pressure, the wound dressing further comprising: a wound contact layer having a first surface for contacting a wound and an opposing second surface; and a covering layer having a first surface facing the wound contact layer and defining a wound dressing cavity and an opposing second surface wherein the wound contact layer is arranged within the cavity; the covering layer further comprising an aperture configured to provide fluid communication between the wound dressing cavity and a source of non-atmospheric pressure.

The tear-drop shape of the wound dressing is particularly suited to application with sources of non-atmospheric pressure (such as in Negative Pressure Wound Therapy), maximising the proportion of the dressing that is provided over the wound whilst allowing the aperture for introducing the of non-atmospheric pressure into the cavity to be isolated from the wound site which benefits the comfort of the patient. By maximising the useable area of the dressing there is a more efficient use of material.

The wound contact layer may have a tear-drop shape comprising a semi-circular portion and a tapered portion.

The central region may be defined as a circular region about the centre point having a radius less than the radius of the semi-circular portion of the wound contact layer. The central region may be defined as a circular region about the centre point having a radius equal to the radius of the semi-circular portion of the wound contact layer.

The aperture may be arranged in the tapered portion of the dressing. The aperture may be arranged outside the central region. The aperture may be provided with a port or airway. The airway may extend away from the tapered end of the wound dressing.

Arranging the aperture in the tapered portion of the dressing, and in particular outside the central region defined as a circular region about the centre point having a radius equal to the radius of the semi-circular portion of the wound contact layer, is considered particularly advantageous. With such an arrangement, the dressing can be arranged with the central region overlying the wound, whilst the tapered portion extends away from the wound, as such the source of negative pressure can be connected away from the wound, such that it is less likely to bear on the wound causing pain. Moreover, particularly in an arrangement where the wound contact layer, absorbent layer and/or transmission layer have a teardrop shaped outline and the adhesive layer/covering layer have an ovate shape, with a constant distance between the outline of the teardrop and the outline of the ovate layer, the ratio of adhesive to non-adhesive region will be highest towards the rounded edge of the tapered portion, and as such, that region - which is at greatest risk of being pulled off, because of the connection to the source of negative pressure should adhere well to the surrounding skin.

The (outer) covering layer of the dressing is provided as a bacterial and viral barrier layer which preferably resists the ingress of liquid and air but allows moisture vapour transmission. In this way the covering layer enhances the overall fluid handling capacity of the dressing by allowing for the escape of moisture vapour through the cover while enabling the application of pressure (either positive or negative) to the wound. The outer covering layer is for instance a layer having a MVTR of at least 3,000 g m⁻² per 24 hours or, more preferably in the range of from 10,000 gm⁻² to 50,000 g m⁻² per 24 hours measured by the method described in BS EN 13726-2 2002 “Test methods for primary wound dressings Part 2 Moisture vapour transmission rate of permeable film dressings”. The covering layer may be in the form of a film of polyurethane, for example Inspire 2350 manufactured by. Alternatively, the covering layer may be in the form of a film of polyethylene.

The wound dressing preferably is a one-piece dressing. That is to say, the covering layer and the body of the wound dressing are provided as an integral item, which the user or HCP is not required to assemble in-situ or before application. The wound dressing preferably including an adhesive layer and preferably including a removable release layer. The wound dressing may be provided in packaging. The packaging may form a sterile cavity in which the wound dressing is provided. The wound dressing may be integrally formed in the packaging.

Providing the wound dressing as a one-piece dressing is advantageous over the more typical NPWT dressing systems which utilise separate packing (for example foam) to contact the wound and drape to cover the wound site and provide a seal for the application of negative pressure. The one-piece system is easier for the user or HCP to apply as the layers are correctly aligned and held together.

The wound dressing may be provided in a kit. The kit may comprise at least one wound dressing and at least one sealing strip. The sealing strips may be shaped to conform to at least a portion of the perimeter of the wound dressing.

In use, the wound dressing may be connected to a source of negative pressure, for example a pump.

Accordingly, in a fourth aspect of the current disclosure there is provided a negative pressure wound therapy system, the system comprising a wound dressing comprising at least one layer having a tear-drop shaped outline, a source of negative pressure and a first length of pressure tubing.

Of course, the wound dressing of the fourth aspect may be the wound dressing of the third aspect, including any optional features thereof.

The source of negative pressure may be a pump. The wound dressing may be in fluid communication with the source of negative pressure via the first length of pressure tubing.

The negative pressure wound therapy system may further comprise an inline filter. The negative pressure wound therapy system may further comprise a second length of pressure tubing. The negative pressure wound therapy system may further comprise a canister. The wound dressing may be in fluid communication with the inline filter via the first length of pressure tubing. The inline filter may be in fluid communication with the source of negative pressure via the second length of pressure tubing. The pressure tubing may be in fluid communication with the cannister.

According to a fifth aspect of the invention there is provided a method of applying a wound dressing comprising at least one layer having a tear-drop shaped outline to a wound site.

Of course, the wound dressing of the fifth aspect may be the wound dressing of the third aspect, including any optional features thereof.

The method may further comprise removing the wound dressing from a sterile packaging. The method may further comprise removing a release layer from the wound dressing. The method may further comprise placing a central region of the wound dressing over the wound site. The method may further comprise positioning the tapered portion away from the wound site.

The method may further comprise connecting the wound dressing to a source of negative pressure.

The present disclosure also provides a wound dressing comprising a wound contact layer and a covering layer. The wound contact layer may have an oval shaped outline. The covering layer may have an oval shaped outline. Preferably both the wound contact layer and the covering layer have an oval shaped outline. The wound dressing may be for use in a pressure gradient wound therapy system. The wound contact layer may have a first surface for contacting a wound and an opposing second surface. The covering layer may have a first surface facing the wound contact layer and an opposing second surface. The first surface of the covering layer may define a wound dressing cavity. The covering layer may comprise an aperture. The aperture may be configured to provide fluid communication between the wound dressing cavity and a pressure gradient source.

According to a sixth aspect of the invention there is provided a wound dressing for use in a pressure gradient wound therapy system, the wound dressing comprising: a wound contact layer having a first surface for contacting a wound and an opposing second surface; and a covering layer having a first surface facing the wound contact layer and defining a wound dressing cavity, and an opposing second surface; the covering layer further comprising an aperture configured to provide fluid communication between the wound dressing cavity and a pressure gradient source; wherein the wound contact layer and the covering layer both have an oval shaped outline.

A wound dressing comprising a wound contact layer and covering layer both with an oval shape can provide an improved application of pressure to the wound site and comfort to the user. The oval shape can allow the dressing to be applied to areas that would not suit a conventional square or rectangular dressing, in particular Breast, sacral, abdominal, spinal and other contoured body parts.

Providing both the covering layer, which defines the overall shape of the dressing, and the wound contact layer, which covers the wound, with the oval shape is especially advantageous compared to dressings having an oval wound contact layer, but a rectangular covering layer, because the oval covering layer is less likely to kink or crease, opening up gaps thorough which air may leak - with a potentially detrimental effect on wound therapy.

The wound contact layer and covering layer may have the same shape, that is they may be geometrically similar, having the same proportions but may have different sizes. For example, the covering layer may be uniformly scaled up with respect to the wound contact layer. The arrangement of the layers may also be the same.

The wound dressing may have a major axis. The major axis of the wound dressing may be the major axis of the oval shape of the wound contact layer. The major axis of the wound dressing may be the major axis of the oval shape of the covering layer. The wound dressing may have a minor axis. The minor axis of the wound dressing may be the minor axis of the oval shape of the covering layer. The oval shape of the wound contact layer may define a major axis. The oval shape of the wound contact layer may define a minor axis. The length of the wound dressing (and each layer thereof) may be defined as the dimension aligned with, for example co-axial with the major axis. The width of the wound dressing (and each layer thereof) may be defined as the dimension aligned with, for example co-axial with the minor axis. The major axis and the minor axis may intersect at a centre point of the wound dressing (the centre point being defined as the centre point of the oval defined by the shape of the wound contact layer). A central region may be defined about the centre point. The centre point may be the centre of a circular central region of the wound dressing. The central region may be defined as a circular region about the centre point having a diameter less than the width of the wound contact layer. The central region may be defined as a circular region about the centre point having a diameter equal to the width of the wound contact layer. The wound dressing may be symmetrical about the major axis. The wound dressing may be symmetrical about the minor axis.

The wound dressing may be provided with two major apexes at the ends of the major axis. The wound dressing may extend along the major axis, beyond the central region to each major apex. The region of the wound dressing which extends from the central region to a first major apex may define a first tapered portion. The region of the wound dressing which extends from the central region to a second major apex may define a second tapered portion.

The wound contact layer may be elliptical. The covering layer may be elliptical.

The wound dressing may comprise a wound-site adhesive layer. The wound dressing may comprise an absorbent layer, for example a superabsorbent layer. The wound dressing may comprise a transmission layer. The wound dressing may comprise a fluid distribution layer, for example a wicking layer. The wound dressing may comprise a pressure distribution layer, for example a foam layer. The wound dressing may comprise a gauze layer. The wound contact layer may be a silicone wound contact layer. One or more or all layers may be joined to the adjacent layer(s). The layers may be joined with an adhesive.

A/the wound-site adhesive layer may have an oval shape. An/the absorbent layer may have an oval shape. A/the transmission layer may have an oval shape. A/the fluid distribution layer may have an oval shape. A/the wicking layer may have an oval shape. An/the absorbent layer may have an oval shape. A/the superabsorbent layer may have an oval shape. A/the foam layer may have an oval shape. A/the pressure distribution layer may have an oval shape. A/the gauze layer may have an oval shape. An/the intralayer adhesive layer may have an oval shape. Every layer of the wound dressing may have an oval shape. Every layer of the wound dressing may have the same centre point. That is every layer of the wound dressing is radially nested. Each layer of the wound dressing may have the same symmetry.

A/the wound-site adhesive layer may have an elliptical shape. An/the absorbent layer may have an elliptical shape. A/the transmission layer may have an elliptical shape. A/the fluid distribution layer may have an elliptical shape. A/the wicking layer may have an elliptical shape. An/the absorbent layer may have an elliptical shape. A/the superabsorbent layer may have an elliptical shape. A/the foam layer may have an elliptical shape. A/the pressure distribution layer may have an elliptical shape. A/the gauze layer may have an elliptical shape. An/the intralayer adhesive layer may have an elliptical shape. Every layer of the wound dressing may have an elliptical shape.

The wound dressing may comprise at least three layers, the covering layer, the wound contact layer and a further layer selected from the following: a wound-site adhesive layer, a fluid distribution layer, a wicking layer, an absorbent layer, a superabsorbent layer, a transmission layer, a foam layer, a pressure distribution layer, a gauze layer and/or an intralayer adhesive layer. Preferably the further layer is an absorbent layer. The further layer may be arranged between the wound contact layer and the covering layer. The further layer may have the same outline as the wound contact layer. The further layer may have an oval shaped outline. The further layer may have an elliptical outline. The wound contact layer and the further layer may be the same size. The wound contact layer and the further layer may be different sizes. The further layer may the larger than the wound contact layer. The further layer may be smaller than the wound contact layer. The wound contact layer and the further layer may have the same centre point. The wound contact layer and the further layer may have the same symmetry. The covering layer may be the same shape as the further layer. The covering layer may be larger than the wound contact layer and/or further layer.

The wound dressing may comprise at least four layers, the covering layer, the wound contact layer and two further layers selected from the following: a wound-site adhesive layer, a fluid distribution layer, a wicking layer, an absorbent layer, a superabsorbent layer, a transmission layer, a foam layer, a pressure distribution layer, a gauze layer and/or an intralayer adhesive layer. Preferably, the two further layers are an absorbent layer and a pressure distribution layer. The further layers may be arranged between the wound contact layer and the covering layer. Where the further layers are an absorbent layer and a pressure distribution layer, the layers may be ordered: the wound contact layer, the absorbent layer, the pressure distribution layer and the covering layer. One or both further layers may have the same outline as the wound contact layer. The two further layers may have the same outline. The two further layers may have a different outline. One or both further layers may have an oval shaped outline. One or both further layers may have an elliptical outline. The wound contact layer and one or both further layers may be the same size. The wound contact layer and one or both further layers may be different sizes. One or both further layers may the larger than the wound contact layer. One or both further layers may be smaller than the wound contact layer. The wound contact layer and one or both further layers may have the same centre point. The wound contact layer and one or both further layers may have the same symmetry. The covering layer may be the same shape as one or both further layers. The covering layer may be larger than the wound contact layer and/or one or both further layers.

The wound dressing may comprise at least five layers, the wound contact layer, covering layer and three layers selected from the following: a wound-site adhesive layer, a fluid distribution layer, a wicking layer, an absorbent layer, a superabsorbent layer, a transmission layer, a foam layer, a pressure distribution layer, a gauze layer or an intralayer adhesive layer. Preferably, the three further layers may be an absorbent layer, a pressure distribution layer and a wound-site adhesive layer. The further layers may be arranged between the wound contacting layer and the covering layer. Where the further layers are an absorbent layer and a pressure distribution layer and wound-site adhesive layer, the layers may be ordered: the wound contact layer, the absorbent layer, the pressure distribution layer, wound-site contact layer and the covering layer. One or more or each further layer may have the same outline as the wound contact layer. Two of the three further layers may have the same outline. The three further layers may have the same outline. The three further layers may have a different outline. One or more or each further layer may have an oval shaped outline. One or more or each further layer may have an elliptical outline. The wound contact layer and one or more or each further layer may be the same size. The wound contact layer and one or more or each further layer may be different sizes. One or more or each further layer may the larger than the wound contact layer. One or more or each further layer may be smaller than the wound contact layer. The wound contact layer and one or more or each further layer may have the same centre point. The wound contact layer and one or more or each further layer may have the same symmetry. The covering layer may be the same shape as the one or more or each further layer. The covering layer may be larger than the wound contact layer and/or one or more or each further layer.

A region of the first surface of the covering layer may be provided with an adhesive. A border region of the first surface of the covering layer may be provided with an adhesive. The adhesive may be provided about a whole perimeter of the covering layer. Where an adhesive is provided on the first surface of the covering layer, the covering layer may also be a wound-site adhesive layer.

A region of the covering layer within the border defined by an/the adhesive border may be closed, sealed and/or uninterrupted. This restricts and or prevents microbes, bacteria and the like from entering the wound site. The wound dressing may comprise a release layer, the release layer being removable to reveal the adhesive border.

The radial extent to which the covering layer extends beyond the outline of the wound contact layer may be constant. The radial extent to which the wound-site adhesive layer extends beyond the outline of the wound contact layer may be constant. The wound-site adhesive layer and wound contact layer may radially overlap such that the wound-site adhesive layer is adhered to the wound contact layer. The radial overlap between the wound contact layer and wound-site adhesive layer may be uniform around the full circumference of the overlap. A radially outer edge of the wound-site adhesive layer may be coterminous with a radially outer edge of the covering layer. A radially inner edge of the wound-site adhesive layer may correspond to a radially outer peripheral edge of the wound contact layer.

The covering layer may extend beyond a perimeter of the wound contact layer. The covering layer may extend an equal distance beyond the perimeter of the wound contact layer about the entire perimeter. An adhesive may be provided on a border of the covering layer. An adhesive may be provided on the portion of the covering layer which extends beyond the perimeter of the wound contact layer. A region of the covering layer extending between a perimeter of the wound contact layer and a perimeter of the covering layer may be provided with an adhesive. An adhesive may be provided on the first surface of the covering layer in the region between the wound contact layer perimeter and the covering layer perimeter. The adhesive may be provided about the entire covering layer perimeter, that is the adhesive forms a continuous closed loop around the wound contact layer.

The wound dressing may comprise a wound contacting layer and a wound-site adhesive layer for adhering the wound dressing to the wound site. The wound-site adhesive layer may extend beyond a perimeter of the wound contact layer. The wound-site adhesive layer may extend an equal distance beyond the perimeter of the wound contact layer about the entire perimeter. An adhesive may be provided on the border of the wound-site adhesive layer. An adhesive may be provided on the portion of the wound-site adhesive layer which extends beyond the perimeter of the wound contact layer.

The wound dressing may have a length of between 100 and 300 mm, preferably between 200 and 300 mm, more preferably between 225 and 375 mm, for example 270 mm. The wound dressing may have a width of between 100 and 250 mm, preferably between 150 and 200 mm, more preferably between 165 and 175, for example 190 mm.

The wound contact layer may have a length of between 100 and 300 mm, preferably between 150 and 250 mm, more preferably between 175 and 225 mm, for example 200 mm. The wound contact layer may have a width of between 80 and 160 mm, preferably between 100 and 140 mm, more preferably between 110 and 130, for example 120 mm.

The border region may have a thickness of between 10 and 50 mm, preferably between 20 and 40 mm, more preferably between 30 and 40 mm, for example 35 mm.

The wound dressing may have a thickness between 1 mm to 20 mm, or 2 mm to 10 mm, or 3 mm to 7 mm, for example.

The wound dressing may be configured for use in negative pressure wound therapy. The wound dressing may be configured for use in positive pressure wound therapy.

When used herein and throughout the specification the term “pressure gradient wound therapy system” is intended to cover a wound therapy system wherein a pressure differential (either positive or negative) is applied between a sealed region of the wound dressing and the surrounding environment.

As used herein, negative pressure wound therapy is a therapeutic technique using a suction dressing to remove excess exudation and promote healing in acute or chronic wounds. A vacuum of −50 to −200 mm Hg, or −75 to −150 mm Hg may be applied with typical negative pressure of −80 to −130 mm Hg, −100 to −130 mm Hg, or often about −125 mm Hg being applied to a wound.

For positive pressure wound therapy, a net positive pressure is applied to the wound, which may include providing simultaneous aspiration and irrigation of the wound. Positive pressure wound therapy may be carried out at a positive pressure of up to 50% atm., typically at a low positive pressure of up to 20% atm., more usually up to 10% atm. At the wound. Positive pressure wound therapy is known and referred to in US20180140755.

The aperture may be arranged outside the central region. The aperture may be arranged on the major axis. The aperture may be provided with a port or airway. The airway may extend away from the central region. The airway may extend away from the central region along a line that extends from the centre point to the aperture, e.g. along the major axis, away from the aperture.

Arranging the aperture outside the central region defined as a circular region about the centre point having a diameter equal to the width of the wound contact layer, is considered particularly advantageous. With such an arrangement, the dressing can be arranged with the central region overlying the wound whilst the remaining portion of the dressing extends beyond from the wound, as such the source of negative pressure can be connected away from the wound, e.g. over the periwound such that it is less likely to bear on the wound causing pain.

Moreover, particularly in an arrangement where the wound contact layer, absorbent layer and/or transmission layer have an oval shaped outline and the adhesive layer/covering layer has a constant distance between the outline of the wound contact layer and the outline of the adhesive layer/covering layer, the ratio of adhesive to non-adhesive region will be highest towards the two apexes of the dressing, and as such, that region—which is at greatest risk of being pulled off, because of the connection to the source of negative pressure should adhere well to the surrounding skin.

The (outer) covering layer of the dressing is provided as a bacterial and viral barrier layer which preferably resists the ingress of liquid and air but allows moisture vapour transmission. In this way the covering layer enhances the overall fluid handling capacity of the dressing by allowing for the escape of moisture vapour through the cover while enabling the application of pressure (either positive or negative) to the wound. The outer covering layer is for instance a layer having a MVTR of at least 3,000 g m⁻² per 24 hours or, more preferably, in the range of from 10,000 gm⁻² to 50,000 g m⁻² per 24 hours measured by the method described in BS EN 13726-2 2002 “Test methods for primary wound dressings Part 2 Moisture vapour transmission rate of permeable film dressings”. The covering layer may be in the form of a film of polyurethane, for example Inspire 2350 manufactured by Coveris. Alternatively, the covering layer may be in the form of a film of polyethylene.

The wound dressing preferably is a one-piece dressing. That is to say, the covering layer and the body of the wound dressing are provided as an integral item, which the user or HCP is not required to assemble in-situ or before application. The wound dressing preferably includes an adhesive layer. The wound dressing preferably includes a removable release layer. The wound dressing may be provided in packaging. The packaging may form a sterile cavity in which the wound dressing is provided. The wound dressing may be integrally formed in the packaging.

Providing the wound dressing as a one-piece dressing is advantageous over the more traditional NPWT dressing systems which utilise separate packing (for example foam) to fill the wound and a drape to cover the wound site and provide a seal for the application of negative pressure. The one-piece system is easier for the user or HCP to apply as the layers are correctly aligned and held together. A one-piece dressing may be better suited for a relatively small, relatively low-exuding wound and a cannister-less NPWT system.

The wound dressing may be provided in a kit. The kit may comprise at least one wound dressing and at least one sealing strip. The sealing strips may be shaped to conform to at least a portion of the perimeter of the wound dressing.

In use, the wound dressing may be connected to a source of negative pressure, for example a pump. Accordingly, the kit may comprise a source of negative pressure, for example a pump, and/or tubing for connecting the wound dressing to the pump.

Accordingly, in a seventh aspect of the current disclosure there is provided a pressure gradient wound therapy system, the system comprising a wound dressing comprising a wound contact layer and a covering layer, both layers having an oval shaped outline, a pressure gradient source and a first length of pressure tubing.

Of course, the wound dressing of the seventh aspect may be the wound dressing of the sixth aspect, including any optional features thereof.

The system may be a negative pressure wound therapy system. The pressure gradient source may be a source of negative pressure. The source of negative pressure may be a pump. The wound dressing may be in fluid communication with the source of negative pressure via the first length of pressure tubing.

The negative pressure wound therapy system may further comprise an inline filter. The negative pressure wound therapy system may further comprise a second length of pressure tubing. The negative pressure wound therapy system may further comprise a canister. The wound dressing may be in fluid communication with the inline filter via the first length of pressure tubing. The inline filter may be in fluid communication with the source of negative pressure via the second length of pressure tubing. One or both lengths of pressure tubing may be in fluid communication with the cannister. Alternatively, the NPWT system may be cannister-less.

According to an eighth aspect of the invention there is provided a method of applying a wound dressing comprising a wound contact layer and a covering layer, both layers having an oval shaped outline to a wound site.

Of course, the wound dressing of the eighth aspect may be the wound dressing of the sixth aspect, including any optional features thereof.

The method may further comprise removing the wound dressing from a sterile packaging. The method may further comprise removing a release layer from the wound dressing. The method may further comprise placing a central region of the wound dressing over the wound site. The method may further comprise positioning one or both tapered portions away from the wound site.

The method may further comprise connecting the wound dressing to a source of negative pressure.

It will be appreciated that, where possible, any feature or combination of features disclosed or claimed herein may be combined with any other feature or combination of features, whether or not this is explicitly described.

### BRIEF DESCRIPTION OF THE DRAWINGS

In order that the invention may be more clearly understood one or more embodiments thereof will now be described, by way of example only, with reference to the accompanying drawings, of which:

FIG. 1 shows a plan view of a dressing according to the present disclosure;

FIG. 2 shows a plan view of the dressing of FIG. 1 with a release layer;

FIG. 3 shows a plan view of another dressing according to the present disclosure;

FIG. 4 shows a further plan view of a kit comprising the dressing of FIG. 3;

FIG. 5 shows a further plan view of a further kit comprising the dressing of FIG. 3;

FIG. 6 shows a further plan view of a further kit comprising the dressing of FIG. 3;

FIG. 7 shows a further plan view of a further kit comprising the dressing of FIG. 3;

FIG. 8 shows a further plan view of a further kit comprising the dressing of FIG. 3;

FIG. 9 shows a further plan view of a further kit comprising the dressing of FIG. 3;

FIG. 10 shows a plan view of another dressing according to the present disclosure;

FIG. 11 shows a plan view of another dressing according to the present disclosure;

FIG. 12 shows a plan view of another dressing according to the present disclosure;

FIG. 13 shows a kit including the dressing of FIG. 12;

FIG. 14 shows a NPWT system including the dressing of FIG. 12.

FIG. 15 shows a plan view of another dressing according to the present disclosure;

FIG. 16 shows an exploded cross sectional view of the dressing of FIG. 15;

FIG. 17 shows a schematic view of a NPWT kit including the dressing of FIG. 15; and

FIG. 18 shows a plan view of the dressing of FIG. 15 with a port attached.

### DETAILED DESCRIPTION OF THE INVENTION

In the following detailed description, numerous specific details are set forth in order to provide a thorough understanding of various embodiments and the inventive concept. However, those skilled in the art will understand that the present invention: may be practiced without these specific details or with known equivalents of these specific details; is not limited to the described embodiments; and may be practiced in a variety of alternative embodiments. It will also be appreciated that well known methods, procedures, components, and systems may have not been described in detail.

Generally, a wound dressing may be taken to be any dressing which is applied to a wound to promote healing and protect against further deterioration of the wound. Wound dressings will typically cover and be held in direct contact with a wound. A wound dressing will generally comprise a wound contacting surface and a rear surface which faces outwards from the wound and defines an exterior of the dressing.

The wound contacting surface may be provided by a separate wound contact layer which has specific properties to promote healing, such as an absorbent pad. Wound contact layers are typically non-adherent layers which are placed in direct contact with the wound and may be designed to promote the removal, e.g. by wicking or absorption, of exudate. A rear surface of the dressing may comprise a separate covering layer to provide the exterior of the dressing and aforementioned rear surface. The covering layer may be configured to provide a sealed envelope against the wound to prevent unwanted contamination of the wound, whilst being optionally breathable to allow moisture egress to aid dressing and wound healing performance. The covering layer may be any suitable material and may, in some embodiments, comprise a polyurethane material, as known in the art and may be LPT 9147 supplied by Covestro AG. The backing layer may have a thickness of between 25 microns and 35 microns, preferably 30 microns.

Wound dressings may be attached to a wound site by any known method of attachment, for example by binding with a bandage or use of adhesive tape, or with some other form of mechanical restraint which holds the dressing in place. In addition, or alternatively, the dressing may comprise a wound-site adhesive layer which is provided on the covering layer or portions of a wound contact layer so as to adhere the dressing to a wound site, as required.

In addition to the wound contact layer, it will be appreciated that many other types of layers may be included in a dressing. A dressing may comprise, for example, a covering layer for covering a wound and may further comprise one or more from the group comprising: a wound contact layer, a fluid distribution layer, a wicking layer, an absorbency layer, a superabsorbency layer, a transmission layer, a foam layer, a gauze layer, a wound-site adhesive layer or intralayer adhesive layer, amongst others. Where a wound dressing comprises a covering layer and one or more other layers, the additional layers will typically be provided in a stacked configuration in which the covering layer is provided as an outer layer which defines an external surface of the dressing, which is typically adhered to the patient covering the wound and providing an envelope in which the other layers can be housed.

Each of the layers may be described in relation to the covering layer and the wound site. Thus, each layer which is located between wound site and covering layer may comprise a first surface which faces inwardly and towards the wound site, and a second surface which faces outwardly from the wound site and towards the backing layer. Similarly, each layer may be described relative to the wound site and the other layers, for example where a second layer is described as being arranged “above” a first layer, the second layer is arranged further from the wound in the stacked configuration with the first later being arranged between.

The wound dressing shape described herein may be used in any type of wound dressing. The specific embodiments described below comprise a covering layer, a wound contact layer, an absorbent pad and a wound-site adhesive layer, but this is not a limitation and one or more of these layers may not be present in some examples. For example, a wound dressing according to the present disclosure may comprise a wound-site adhesive layer and absorbent pad only, in such instances the absorbent pad would also be the wound contact layer. In other embodiments described below, the wound dressing may not comprise an adhesive layer but be attached to a patient using a secondary means, such as the aforementioned bindings or the like.

Wound contact layers and absorbent pads are generally well known in the art and any suitable wound contact layer or absorbent pad may be used. An example of a suitable wound contact layer may be that described in EP2498829 which discloses a wound dressing having an absorbent component for a wound contact layer. The wound contact layer or absorbent pad may comprise gel forming fibres which are bound to a foam layer to aid the absorption of wound exudate, however, this is not a limitation, and any type of wound contact layer or absorbent pad may be used.

The wound dressings of the present disclosure are shaped to lend themselves to application on multiple different types of wound sites whilst having improved conformity to variably or highly contoured parts of the body. In doing so, the wound dressings have increased utility and require less specialist knowledge to be applied. In providing a multi-site dressing shape, it is the intention to provide a dressing which balances ease of use with increased performance in terms of wear-time and patient comfort in numerous different circumstances.

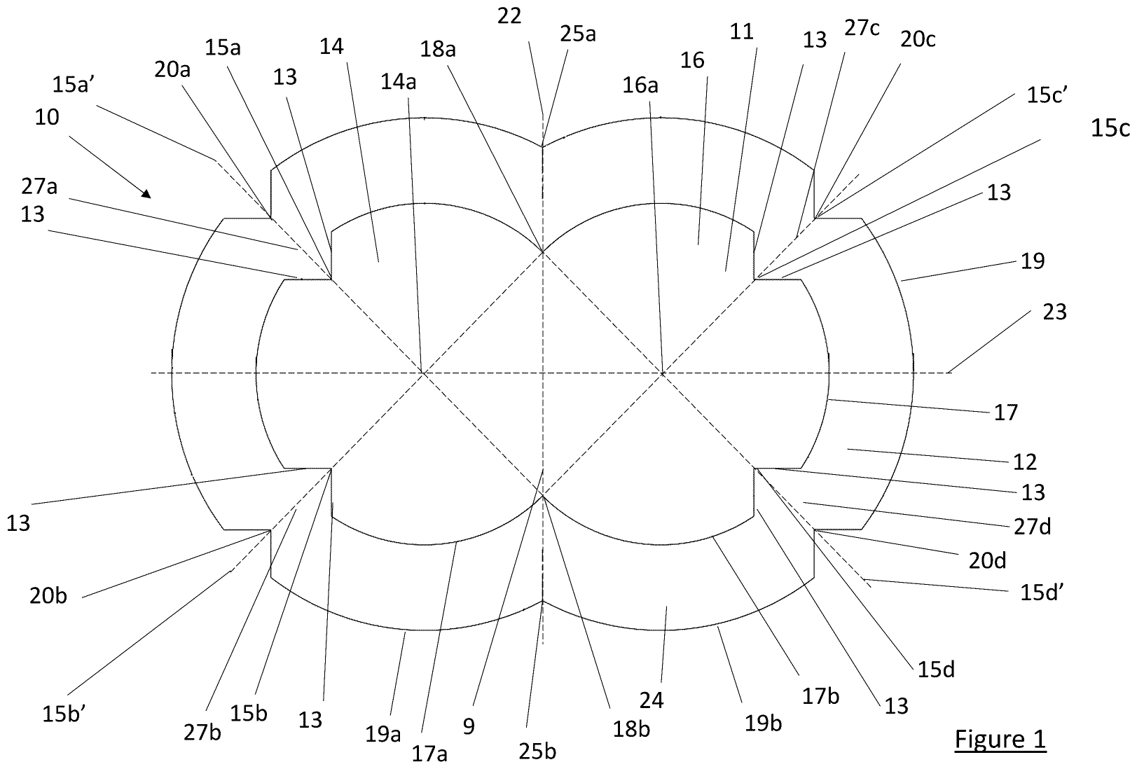

FIG. 1 shows a wound dressing according to the present disclosure. The dressings are considered to be favourable for attaching or adhering to multiple different sites on the human body with variably or highly contoured aspects which may typically be difficult areas to adhere to. These areas may include a shoulder, armpit, elbow, wrist, sacral region, breast, hip and heel, for example.

FIG. 1 shows a wound dressing 10 in plan. The dressing 10 comprises an absorbent pad 11 having a shape comprising two contiguous lobes 14, 16. To aid in the description of the dressing shape, there is shown a centre line 22 which extends along a boundary 9 between the two lobes 14, 16, a second, major axis, line 23 is provided at the midpoint of and perpendicular to the centre line 22, the major axis line 23 extends along a line of symmetry of the dressing 10.

In this embodiment the two lobes 14, 16 are of equal size and have a generally circular shape with each lobe having a respective centre point 14a, 16a, the centre points 14a, 16a being the origin for the circular shape of each lobe 14, 16. The two lobes overlap such that a segment of each defined by an arc and chord with a central angle of 90 degrees is omitted from each lobe. That is, a quarter of the circumference of the circles of each lobe overlaps with the other lobe, such that each lobe extends around 270 degrees of a circle, the absorbent pad 11 thus having a broadly “figure of 8” shape. The peripheral edges of each lobe 17a, 17b meet at two inflection points 18a, 18b, the peripheral edges 17 curving about the respective centre points 14a, 16a until the two peripheral edges 17 meet at an abrupt interface. In other embodiments (which would be asymmetric about the flex line) the two lobes may have different diameters.

In this embodiment each lobe 14, 16 is provided with two notches 15a, 15b, 15c, 15d. To further assist in the description of the dressing shape, FIG. 1 is also provided with notch bisecting lines 15a′ 15b′ 15c′ 15d′, these lines bisect their respective notch 15a, 15b, 15c, 15d. With reference to the first lobe 14, the two notches 15a, 15b are arranged opposing the boundary 9 between the two lobes and symmetrically about the major axis line 23. The angle between the first notch bisecting line 15a′ and the major axis line 23 is 45 degrees about the centre point 14a of the lobe 14. In other embodiments differing numbers of notches may be provided and/or the position of the notches may be varied to various contoured aspects of a wound as will be expanded upon below.

The notches 15 are V-shaped indents in the periphery of the absorbent pad 11, in this embodiment the notches have two straight edges 13 with an internal angle of 90 degrees between them. The notches 15 extend radially inward from the peripheral edge 17 of the absorbent pad 11 one fifth of the way towards the centre point 14a, 16a of their respective lobe 14, 16.

Arranged above the absorbent pad 11 is a covering layer 12, the covering layer 12 is provided with a peripheral edge 19 that has the same shape as the peripheral edge 17 of the absorbent pad 11 but is larger, a uniform scaling having been applied, the covering layer peripheral edge 19 is located radially outwards of the absorbent pad peripheral edge 17, the absorbent pad and covering layer are thus geometrically similar. For each lobe the radial distance, as measured from the centre of said lobe, by which the covering layer peripheral edge 19 extends beyond the absorbent pad peripheral edge 17 remains constant, providing a uniform perimeter to the wound dressing 10. The region between the covering layer peripheral edge 19 and the absorbent pad peripheral edge 17 may define a border region 24.

Because the shape of the covering layer 12 is the same as the shape of the pad, as with the absorbent pad peripheral edges 17a 17b, the covering layer peripheral edges 19a 19b meet at two inflection points 25a 25b. The covering layer inflection points 25 are aligned with the absorbent pad inflection points 18, that is the covering layer inflection points are arranged outward of the absorbent pad inflection points 18, along the flex line.

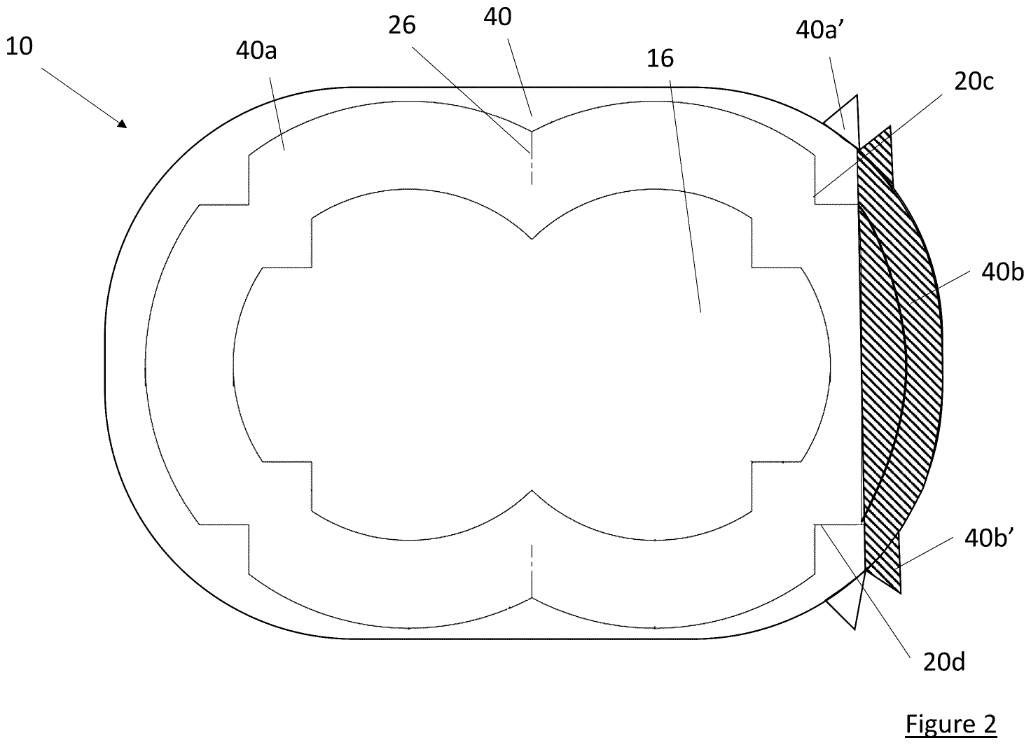

As can be seen in FIG. 2, in some embodiments the border region 24 is provided with two lines of perforations 26 extending between the corresponding sets of inflections points 18, 25. The perforations 26 may extend through the entire border region 24 between the inflection points 18, 25 or as in this embodiment may only extend a portion of the distance from the covering layer peripheral edge 19 to the absorbent pad peripheral edge 17. Alternatively in place of the perforations 26 a slit may be provided. In further alternatives, there may be provided a combination of a slit and perforations. For example, a slit may extend from the covering layer inflection point 19 halfway to the absorbent pad inflection point 18, the remaining distance provided with a line of perforations. The perforations 26 and/or slits provide lines where the border region 24 of the covering layer 12 can be preferentially broken to improve the conformity of the dressing and allow the border region to overlapped to remove bulges in the border region 24.

The covering layer 12 is also provided with four notches 20a, 20b, 20c, 20d the covering layer notches 20 have the same profile and are the same size as the absorbent pad notches 15. Therefore, the covering layer notches 20 do not extend proportionately as far radially toward the lobe centres 14a, 16a. The covering layer notches 20 are aligned with the absorbent pad notches 15, that is to say, each absorbent pad notch 15 is aligned with a covering layer notch 20 wherein a single line 15a′, 15b′, 15c′, 15d′ bisects both the absorbent pad notch 15 and the covering layer notch 20, each aligned absorbent pad notch and the covering layer notch (i.e. 15a, 20a; 15b, 20b; 15c, 20c; and 15d, 20d) form a notch pair 27a, 27b, 27c, 27d.

The notches 15, 20 and inflection points 18, 25 provide preferential points from which flex or fold lines in the wound dressing can be formed so as to fit a contoured wound site. A flex line 22 extends between the two covering layer inflection points 25 along the boundary of the two lobes 14, 16. This flex line 22 allows the wound dressing 10 to be folded to accommodate a ridge or contour in the wound site and thus ensure good adhesion to a non-flat wound site.

Flex lines can also be preferentially formed from one or more of the notch pairs 27, the line 15a′, 15b′, 15c′, 15d′ bisecting the notch pair being a notch flex line about which the wound dressing 10 can also be folded.

Due to the alignment of the flex line and notch flex lines, they do not converge at a single point. The notch flex lines 15a′ 15b′ converge in the centre 14a of the first lobe 14 and intersect with the flex line 26 at opposing absorbent pad inflection points 18. Similarly, the notch flex lines 15c′ 15d′ converge in the centre 16a of the first lobe 16 and intersect with the flex line 22 at opposing absorbent pad inflection points 18. The intersections of the flex line and notch flex lines are at angles of 45 degrees.

By arranging the flex line 22 and notch flex lines 15a′, 15b′, 15c′, 15d′ so that they do not all converge at a single point of the dressing the integrity of the dressing 10 is improved. When the dressing 10 is folded the layers 11, 12 of the dressing become non-planar, when more than one fold line intersects the layers can be drawn apart at the intersection point. This can result in separation of the layers and/or loss of contact between the wound site and a wound contact layer which may affect the ability of the dressing to assist in healing. Furthermore, having notch flex lines intersect in their respective lobes 14, 16 allows the lobes to be conformed to the wound site topography independently. The arrangement whereby each notch flex line intersects with the flex line at a diametrically opposing pad inflection point 18, also aids the folding of the dressings about the notch flex lines, and in particular, means that the folding of one lobe along a notch flex line does not affect the shape of the other lobe (since the fold line does not extend into the other lobe). Having the notches on the periphery of the wound dressing 10 ensures not only that the dressing can fold to conform to a wound site but also that when doing so the periphery does not bulge and lose contact with the wound site. This is important because a bulge in the adhesive layer could provide a channel by which contamination is introduced into the wound site and a weak point in the adhesive causing premature loss of the dressing.

The provision of notches 15, 20 further improves the conformality of the wound dressing 10 over contours on the wound site. Due to the absence of material in the notch, the two edges 13 of an absorbent pad notch 15 and/or adhesive layer notch 20 can be brought together giving the dressing a shallow conical shape. This can be particularly useful where the dressing 10 is applied to heels, knees, elbows, etc.

As noted above in different embodiments alternative arrangements and alignment of notches may be provided for, allowing the dressing to be conformed to particular wound site contours. For example, each lobe may be provided with four notches allowing the lobe to conform to even more highly contoured wound sites.

Thus, the skilled person will understand that notches can be provided at any point on the peripheral edge of the dressing, the absorbent pad 11 and/or covering layer 12 to provide a terminus for a flex or fold line and thus used to tailor the shape to which the wound dressing 10 can be folded.

In some embodiments the first surface of the covering layer 20 may be provided with an adhesive band (not shown) in the border region 24. As such the covering layer may also be a wound-site adhesive layer. Where an adhesive band is provided the wound dressing 10 may be provided with a release liner 40 arranged to cover the adhesive band until use as can be seen in FIG. 2.

FIG. 2 shows a further plan view of the wound dressing 10, in this instance with a release liner 40, the release liner 40 is formed of two portions, a main release liner portion 40a, and a second release liner portion 40b. The release liner 40 has a generally oblong or stadium shape, and covers and slightly overhangs the border region 24. The second release liner portion 40b is arranged on one lobe 16 at the distal end of the major axis between the two notches 20c, 20d the boundary with the main release liner portion 40a being perpendicular to the major axis. The main release liner 40a covers the remainder of the border region 24. Where the two release liner portions 40a, 40b meet, each is provided with a release tab 40a′, 40b′ which is a continuation of the release liner 40a, 40b not in contact with the adhesive and provided to allow easy removal of the remaining release liner.

To apply the wound dressing 10, the user or health care professional (HCP) can remove the dressing 10 from packaging (not shown) that ensures the sterility of the dressing. With the release layer 40 still attached (if present) the wound dressing 10 can be placed against the wound and the orientation varied to determine the optimal orientation to conform to the wound site contours. Initial folding of the dressing can also be performed at this time. The release layer 40 is then removed, the second release liner portion 40b being removed first by pulling on the corresponding release tab 40b′. A portion of the dressing is then applied to the wound site and then the main release liner portion 40a is removed progressively by pulling on the corresponding release tab 40a′. As the main release liner portion 40a is progressively removed the user or HCP can shape the dressing by folding it about flex lines between notches 15, 20 and/or by bringing the edges 13 of the notches 15 together.

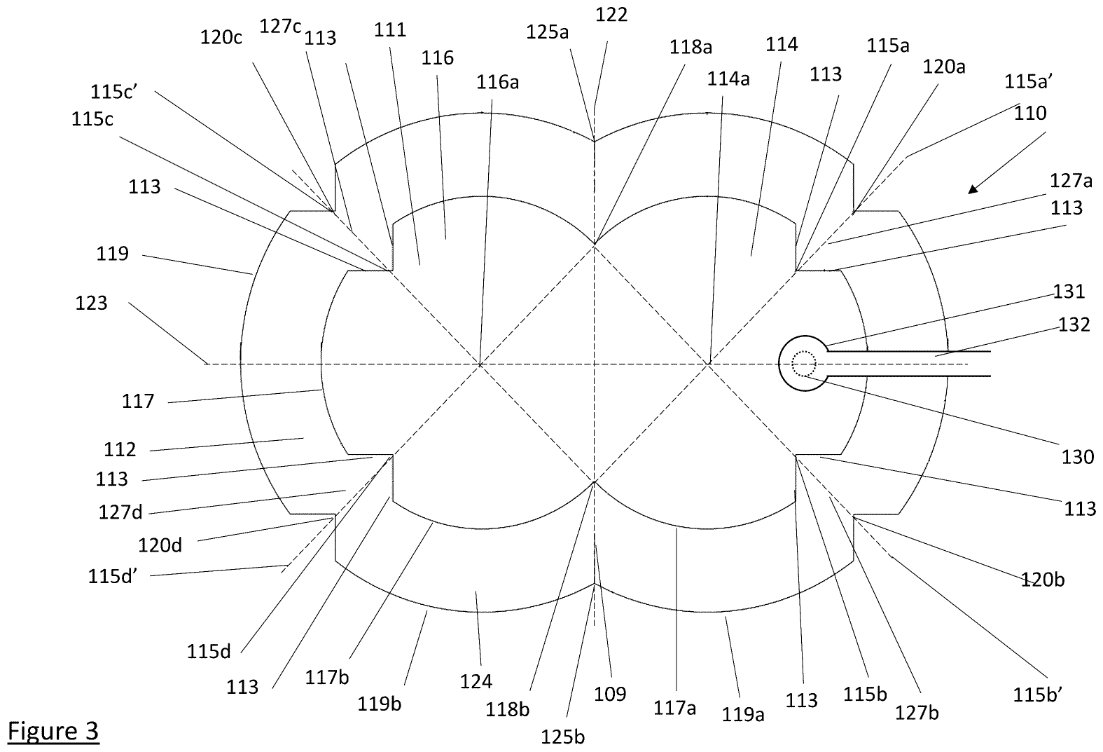

FIG. 3 shows an alternative wound dressing 110 according to the present disclosure. The wound dressing 110 of this embodiment is similar to the wound dressing 10 of the previous embodiment with like features provided with reference numerals advanced by 100. In this embodiment the wound dressing is configured for use with a source of non-atmospheric pressure (not shown), for example for use in pressure gradient wound therapy as is known in the art. The dressings are considered to be favourable for attaching or adhering to multiple different sites on the human body with variably or highly contoured aspects which may typically be difficult areas to adhere to. These areas may include a shoulder, armpit, elbow, wrist, sacral region, breast, hip and heel, for example.

FIG. 3 shows the wound dressing 110 in plan. The dressing 110 comprises an absorbent pad 111 having a shape comprising two contiguous lobes 114, 116. To aid in the description of the dressing shape, there is shown a centre line 122 which extends along a boundary 109 between the two lobes 114, 116, a second, major axis, line 123 is provided at the midpoint of and perpendicular to the centre line 122, the major axis line 123 extends along a line of symmetry of the dressing 110.

In this embodiment the two lobes 114, 116 are of equal size and each have an generally circular shape with each lobe having a respective centre point 114a, 116a. The two lobes 114, 116 overlap such that a segment of each defined by an arc and chord with a central angle of 90 degrees is omitted from each lobe 114, 116, that is a quarter of the circumference of the circles of each lobe overlaps with the other lobe, such that each lobe extends around 270 degrees of a circle, the absorbent pad 111 thus having a broadly “figure of 8” shape. The peripheral edges of each lobe 117a, 117b meet at two inflection points 118a, 118b, the peripheral edges curving about the respective centre points 114a, 116a until the two peripheral edges 117a, 117b meet at an abrupt interface. In other embodiments (which would be asymmetric about the flex line) the two lobes may have different diameters.

In this embodiment each lobe 114, 116 is provided with two notches 115a, 115b, 115c, 115d. To further assist in the description of the dressing shape, FIG. 3 is also provided with notch bisecting lines 115a′, 115b′, 115c′, 115d′, these lines bisect their respective notch 115a, 115b, 115c, 115d. With reference to the first lobe 114, the two notches 115a, 115b are arranged opposing the boundary 113 between the two lobes and symmetrically about the major axis line 123 the angle between the first notch bisecting line 115a′ and the major axis line 123 is 45 degrees about the centre point 114a of the lobe 114. In other embodiments differing numbers of notches may be provided and/or the position of the notches may be varied to allow the dressing 110 to conform to differently contoured aspects of a wound as will be expanded upon below.

The notches 115 are V-shaped indents in the periphery of the absorbent pad 111, in this embodiment the notches have two straight edges 113 with an internal angle of 90 degrees between them. The notches 115 extend radially inward from the peripheral edge 117 of the absorbent pad 111 one fifth of the way towards the centre point 114a, 116a of their respective lobe 114, 116.

Arranged above the absorbent pad 111 is a covering layer 112, the covering layer 112 is provided with a peripheral edge 119 that has the same shape as the peripheral edge 117 of the absorbent pad 111 but is larger, being uniformly scaled up, the covering layer peripheral edge 119 is located radially outwards of the absorbent pad peripheral edge 117, the absorbent pad and covering layer are thus geometrically similar. For each lobe the radial distance, as measured from the centre of said lobe by which the covering layer peripheral edge 119 extends beyond the absorbent pad peripheral edge 117 remains constant, providing a uniform perimeter to the wound dressing 110. The region between the covering layer peripheral edge 119 and the absorbent pad peripheral edge 117 may define a border region 124.

The covering layer 110 of the dressing is provided as a bacterial and viral barrier layer which preferably resists the ingress of liquid and air but allows moisture vapour transmission. In this way the cover layer enhances the overall fluid handling capacity of the dressing by allowing for the escape of moisture vapour through the cover while enabling the application of pressure (either positive or negative) to the wound. The outer cover layer is for instance a layer having a MVTR of at least 3,000 g m⁻² per 24 hours or preferably, in the range of from 10,000 gm⁻² to 50,000 g m⁻² per 24 hours measured by the method described in BS EN 13726-2 2002 “Test methods for primary wound dressings Part 2 Moisture vapour transmission rate of permeable film dressings”. The cover layer may be in the form of a film of polyurethane, for example Inspire 2350 manufactured by Coveris

Because the shape of the covering layer is the same as the shape of the pad, as with the absorbent pad peripheral edges 117a, 117b the covering layer peripheral edges 119a, 119b meet at two inflection points 125a, 125b. The covering layer inflection points 125 are aligned with the absorbent pad inflection points 118, that is the covering layer inflection points 125 are arranged radially outward of the absorbent pad inflection points 118, along the flex line.

The covering layer 112 is also provided with four notches 120a, 120b, 120c, 120d, in this embodiment the covering layer notches 120 have the same profile and are the same size as the absorbent pad notches 115. Therefore, the covering layer notches 120 do not extend proportionately as far radially toward the lobe centres 114a, 116a. The covering layer notches 120 are aligned with the absorbent pad notches 115, that is to say, each absorbent pad notch 115 is aligned with a covering layer notch 120 wherein a single line 115a′, 115b′, 115c′, 115d′ bisects both the absorbent pad notch 115 and the covering layer notch 120 each aligned absorbent pad notch and the covering layer notch (i.e. 115a, 120a; 115b, 120b; 115c, 120c; and 115d, 120d) form a notch pair 127a, 127b, 127c, 127d.

The covering layer 112 is provided with an aperture 130 to allow a source of non-atmospheric pressure (not shown) to be applied to the wound via a port 131 and tubing 132.

In use the notches 115, 120 and inflection points 118, 125 provide preferential points from which flex or fold lines in the wound dressing 110 can be formed so as to fit a contoured wound site. A flex line 126 extends between the two covering layer inflection points 125 along the boundary of the two lobes 114, 116. This flex line 126 allows the wound dressing 110 to be folded to accommodate a ridge or contour in the wound site and thus ensure good adhesion to a non-flat wound site and thus an airtight seal for the application of non-atmospheric pressure.

Flex lines can also be preferentially formed from one or more of the notch pairs 127, the line 115a′, 115b′, 115c′, 115d′ bisecting the notch pair being a notch flex line about which the wound dressing 110 can also be folded.

Due to the alignment of the flex line and notch flex lines, they do not converge at a single point. The notch flex lines 115a′ 115b′ converge in the centre 114a of the first lobe 114 and intersect with the flex line 126 at opposing absorbent pad inflection points 118. Similarly, the notch flex lines 115c′ 115d′ converge in the centre 16a of the first lobe 116 and intersect with the flex line 126 at opposing absorbent pad inflection points 118. The intersections of the flex line and notch flex lines are at angles of 45 degrees.

By arranging the flex line 126 and notch flex lines 115a′, 115b′, 115c′, 115d′ so that they do not all converge at a single point of the dressing the integrity of the dressing 110 is improved. When the dressing 110 is folded the layers become non-planar, when more than one fold line intersects the layers can be drawn apart at the intersection point. This can result in separation of the layers and/or loss of contact between the wound site and the wound contact layer which may affect the ability of the dressing to assist in healing. It is particularly beneficial when the dressing. Furthermore, having notch flex lines 115a′, 115b′, 115c′, 115d′ intersect in their respective lobes 114, 116 allows the lobes to be conformed to the wound site topography independently.

The arrangement whereby each notch flex line intersects with the flex line at an inflection point 118, also aids the folding of the dressings about the notch flex lines, and in particular, means that the folding of one lobe along a flex line does not affect the shape of the other lobe (since the fold line does not extend into the other lobe).

In addition to providing points from which a fold may be preferentially formed, the provision of notches 115, 120 further improves the conformality of the wound dressing 110 over contours on the wound site. due to the absence of material within the notch, the two edges 113 of an absorbent pad notch 115 can be brought together giving the dressing a shallow conical shape. This can be particularly useful where the dressing 110 is applied to heels, knees, elbows, etc.

Having the notches on the periphery of the wound dressing 110 ensures not only that the dressing can fold to conform to a wound site but also that when doing so the periphery does not bulge and come out of contact with the wound site. This is important because a bulge in the adhesive layer could provide a channel by which contamination can be introduced into the wound site and a weak point in the adhesive causing premature loss of the dressing. A particular benefit of notches in the periphery of the dressing in the context of non-atmospheric pressure treatment is that it improves the seal provided by the adhesive layer as bulges could introduce leaks hindering treatment.

The method of applying the wound dressing 110 of this embodiment is the same as that of the previous embodiment, the user or health care professional (HCP) can remove the dressing 110 from packaging. With the release layer still attached the wound dressing 110 can be placed against the wound and the orientation varied to determine the optimal orientation to conform to the wound site contours. Initial folding of the dressing can also be performed at this time. The release layer is then removed the second release liner portion being removed first by pulling on the corresponding release tab. A portion of the dressing is then applied to the wound site and then the main release liner portion is removed progressively by pulling on the corresponding release tab. As the main release liner portion is progressively removed the user of HCP can shape the dressing by folding it about flex lines between notches 115, 120 and/or by bringing the edges 113 of the notches 115 together.

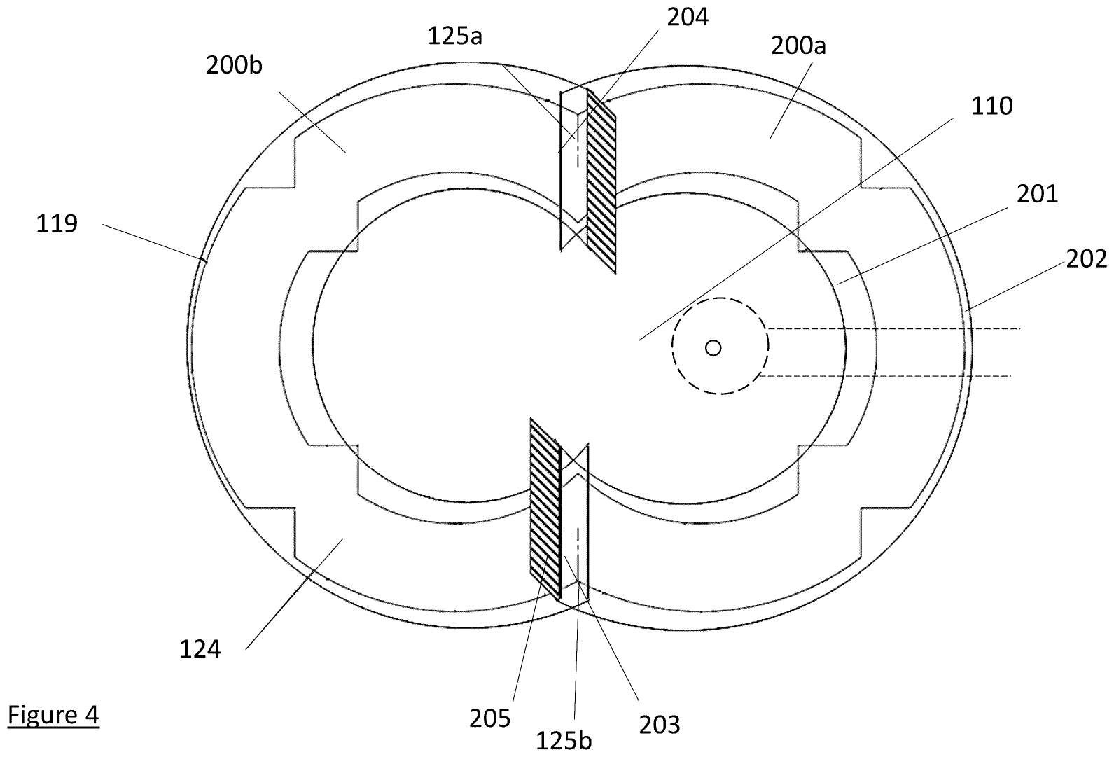

In some embodiments the wound dressing 110 may be supplied as part of a kit containing other components required for NPWT such as a port, tubing and one or more sealing strips. The one or more sealing strips 200, 300, 400, 500, 600, 700 are provided to improve the seal between the dressing 110 and the wound site about the border region 124. With reference to FIGS. 4 to 10 the sealing strips 200, 300, 400, 500, 600, 700 are shown in greater detail below.

FIG. 4 shows a plan view of the wound dressing 110 configured for use with a source on non-atmospheric pressure. The wound dressing 110 is provided in a kit with two sealing strips 200a,b. The two sealing strips 200a,b have the same shape, as such only one will be described in detail. The sealing strip 200a is formed from an annular segment of adhesive tape having an adhesive on one side. The sealing strip 200a has two curved edges, a radially inner edge 201 and a radially outer edge 202, both have a circular profile with a common centre point. Completing the perimeter of the strip 200a are a first end 203 and a second end 204, both ends 203,204 are arranged at an acute angle relative to the radially inner edge 201. The first end 203 is provided with a release tab 205 to aid in application. The sealing strip 200a has an overall “U” shape. The sealing strip 200a has a constant width along its length, the width being greater than that of the border region 124 of the dressing such that in use the sealing strip 201 extends beyond the peripheral edge of the covering layer 119.

In use, once the wound dressing 110 has been applied to the wound site the user or HCP places the sealing strips 200a,b sequentially over the border region 124 such that it overlaps and extends beyond the peripheral edge 119 of the covering layer. Each strip 200 extends from one inflection point 125a in the covering layer to the opposite covering layer inflection point 125b thereby sealing a complete lobe 114, 116. At the inflection points 125 the two sealing strips 200a,b slightly overlap to ensure the dressing is sealed around its entire peripheral edge 119.

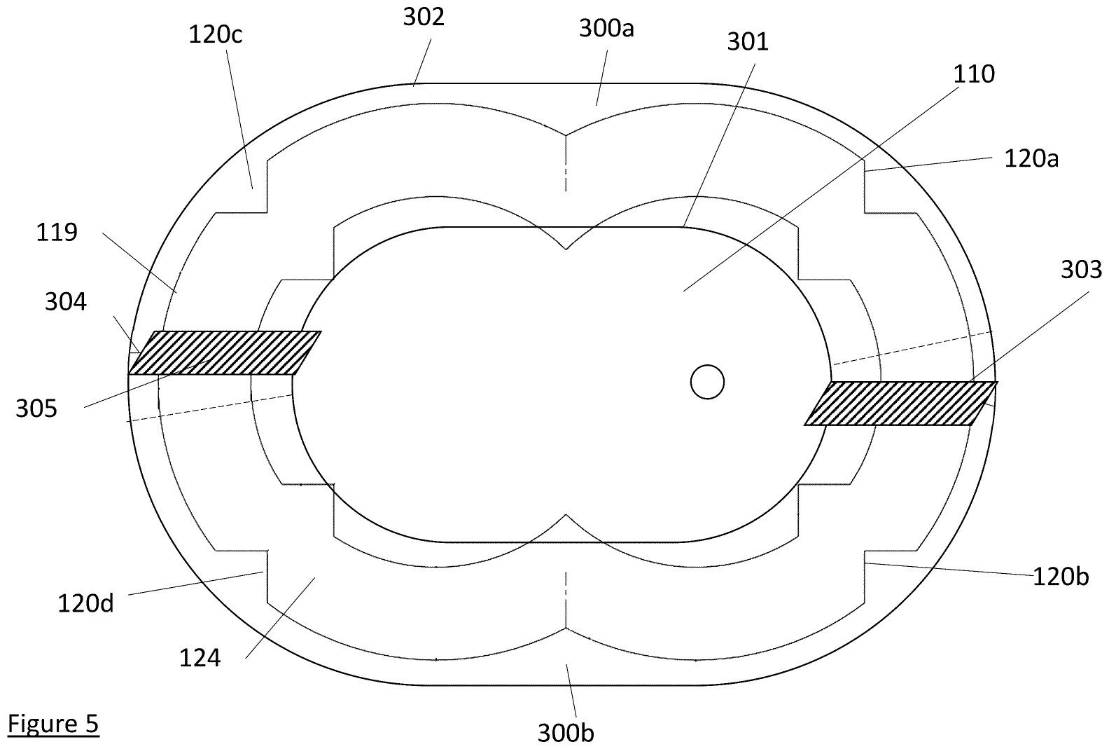

FIG. 5 shows an alternative kit of wound dressing 110 and sealing strips 300. In this kit there are again provided two sealing strips 300a,b. The two sealing strips have the same shape, as such only one will be described in detail. The sealing strip 300a is formed from an annular segment of adhesive tape having an adhesive on one side. The sealing strip 300a has two curved edges, an inner edge 301 and an outer edge 302. Completing the perimeter of the strip 300a are a first end 303 and a second end 304, both ends 303,304 are arranged perpendicular to the inner edge 201. The sealing strip 300 has an overall “C” shape. The sealing strip 300 has a constant width along its length, the width being greater than that of the border region 124 of the dressing such that in use the sealing strip 300 extends beyond the peripheral edge of the covering layer 119. The second end 304 is also provided with a release tab 305.

In use, once the wound dressing 110 has been applied to the wound site the user or HCP places the sealing strips 300 sequentially over the border region 124 such that it overlaps and extends beyond the peripheral edge 119 of the covering layer 112. Each strip 300 extends from a mid-point between the two covering layer notches 120c,d in a lobe 116 about the peripheral edge 119 of the dressing 110 to a mid-point between the two covering layer notches 120a,b in the opposite lobe 114. At the mid-points the two sealings strips overlap to ensure a complete seal around the entire periphery 119.

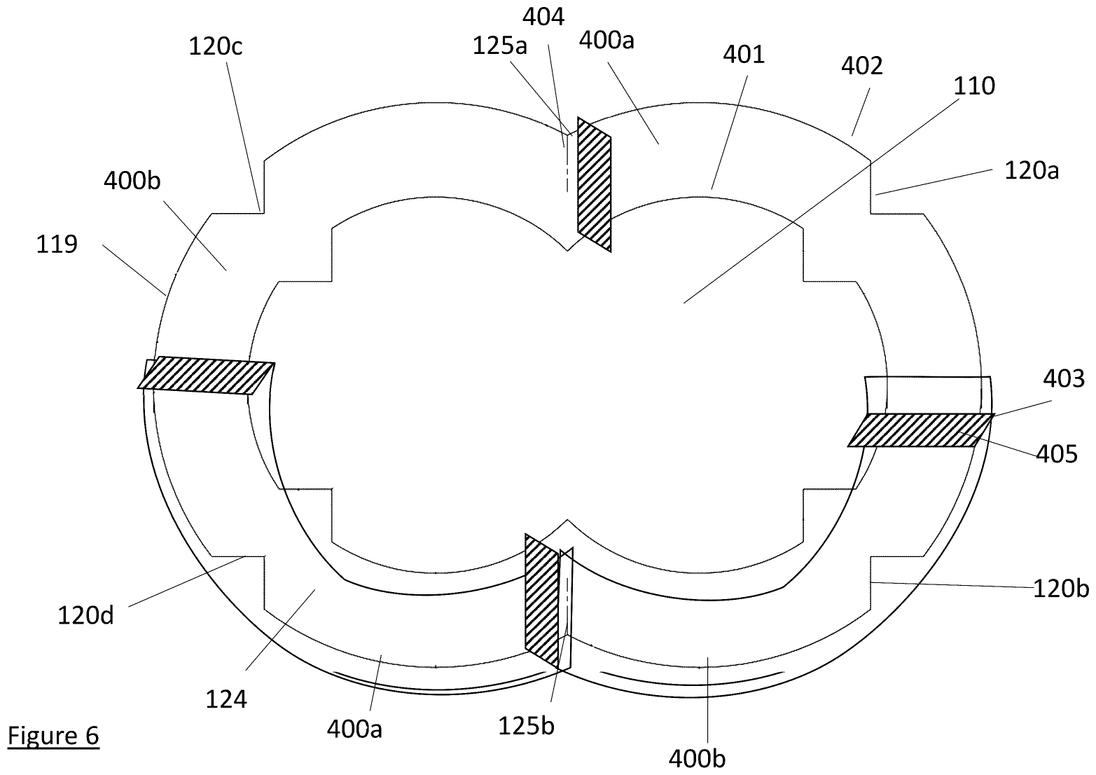

FIG. 6 shows an alternative kit of wound dressing 110 and sealing strips 400. In this kit there are provided four sealing strips 400. The four sealing strips are provided as two strips of a first shape 400a and two strips of a second shape 400b. The two shapes 400a, 400b are congruent, one being a reflection of the other, as such only one sealing strip is described in detail. The sealing strip 400a is formed from an annular segment approximately one third of a complete annulus having a radially inner edge 401 and a radially outer edge 402, both edges 401, 402 have a common centre point. A first end 403 of the sealing strip 400a is perpendicular to both the radially inner 401 and outer 402 edges of the strip. A second end 404 of the sealing strip 400a is provided at an acute angle relative to the radially inner edge 401. The first end 403 is also provided with a release tab 405 which facilitates the removal of a release layer on the sealing strip 400a prior to application. The sealing strip 400a is an adhesive tape having an adhesive on one side. The sealing strip 400a has a constant width along its length, the width being greater than that of the border region 124 of the dressing such that in use the sealing strip 400a extends beyond the peripheral edge of the covering layer 119. As noted above the two shapes of sealing strip in this embodiment are congruent, the only difference being that the two ends 403, 404 are swapped on the second sealing strips 400b.

In use, once the wound dressing 110 has been applied to the wound site the user or HCP places the sealing strips 400 sequentially over the border region 124 such that it overlaps and extends beyond the peripheral edge 119 of the covering layer 112. One of the first shape strips 400a extends from a midpoint between the covering layer notches 120a,b in a first lobe 114 to the first inflection point between the two lobes 125a on the peripheral edge 119 of the covering layer, the second end 403 is arranged at the inflection point. One of the second shape strips 400b is then arranged such that it extends between the first inflection point 125a and the mid-point between the two notches 120c,d in the second lobe 116, the two sealing strips 400a,b overlapping slightly at the inflection point 125a, again the second end 403 is arranged at the inflection point 125a. A further first shape is then arranged to extend between the mid-point between the two notches 120c, d in the second lobe 116 and the second inflection point 125b, the sealing strip overlapping with the second shape strip 400b, again the second end 403 is arranged at the second inflection point 125b. Another second shape strip 400b is then arranged between the second inflection point 125b and the midpoint between the two covering layer notches 120a.b in the first lobe 114. Again, the second end 404 is arranged at the second inflection point 125b and the two ends 403,404 overlap with the neighbouring strips. The sealing strips increase the adhesive area of the dressing improving the seal.

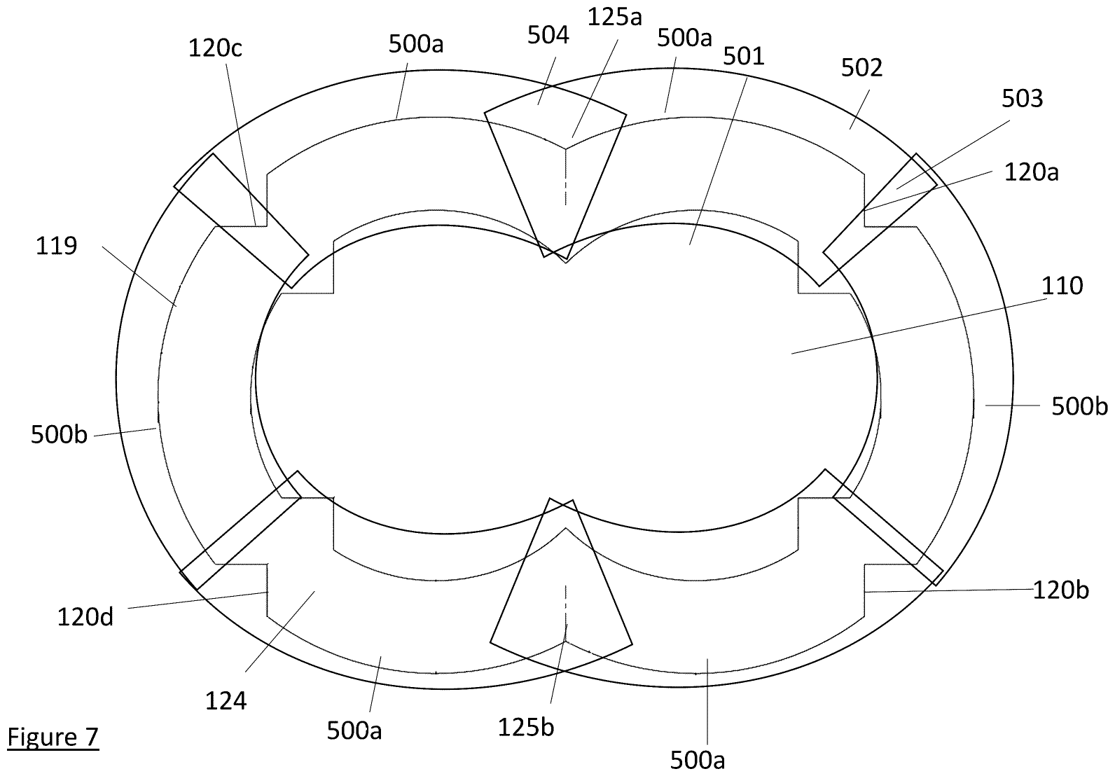

FIG. 7 shows an alternative kit of wound dressing 110 and sealing strips 500. In this kit there are provided six sealing strips 500. The six sealing strips 500 are provided as four strips of a first shape 500a and two strips of a second shape 500b. The two shapes 500a, 500b differ only in that the second shape strips 500b are narrower, as such only one sealing strip is described in detail. The first shape sealing strip 500a is formed from an annular segment approximately one sixth of a complete annulus having a radially inner edge 501 and a radially outer edge 502, both edges 501, 502 have a common centre point. Each end 503, 504 of the sealing strip 500a is perpendicular to both the radially inner 501 and outer 502 edges of the strip. The sealing strip 500a is an adhesive tape having an adhesive on one side. The sealing strip 500a has a constant width along its length, the width being greater than that of the border region 124 of the dressing such that in use the sealing strip 500a extends beyond the peripheral edge of the covering layer 119. As noted above the two shaped of sealing strip in this embodiment are the same, the only difference being that the width of the second shape sealing strips 500b is less than that of the first shape sealing strips.

In use, once the wound dressing 110 has been applied to the wound site the user or HCP places the sealing strips 500 sequentially over the border region 124 such that it overlaps and extends beyond the peripheral edge of the covering layer. One of the first shape strips 500a is placed such that it extends between the covering layer notches 120a,b in a first lobe. One of the second shape strips 500b is then arranged such that it extends between a notch 120a in the first lobe and the first inflection point 125a. A further second shape sealing strip 500b is then arranged to extend between the first inflection point 125a and the covering layer notch 120c on the second lobe. A first shape sealing strip 500a is then arranged to extend between the two covering layer notches 120c,d on the second lobe 116. A further second shape sealing strip 500b is then arranged to extend between the covering layer notch 120d in the second lobe and the second inflection point 125b. Another second shape sealing strip 500b is then arranged to extend between the second inflection point 125b and the covering layer notch 120b where one end of the first placed sealing strip is already in place. As in the previous embodiments, each sealing strip 500 slightly overlaps with the neighbouring strips to ensure an airtight seal.

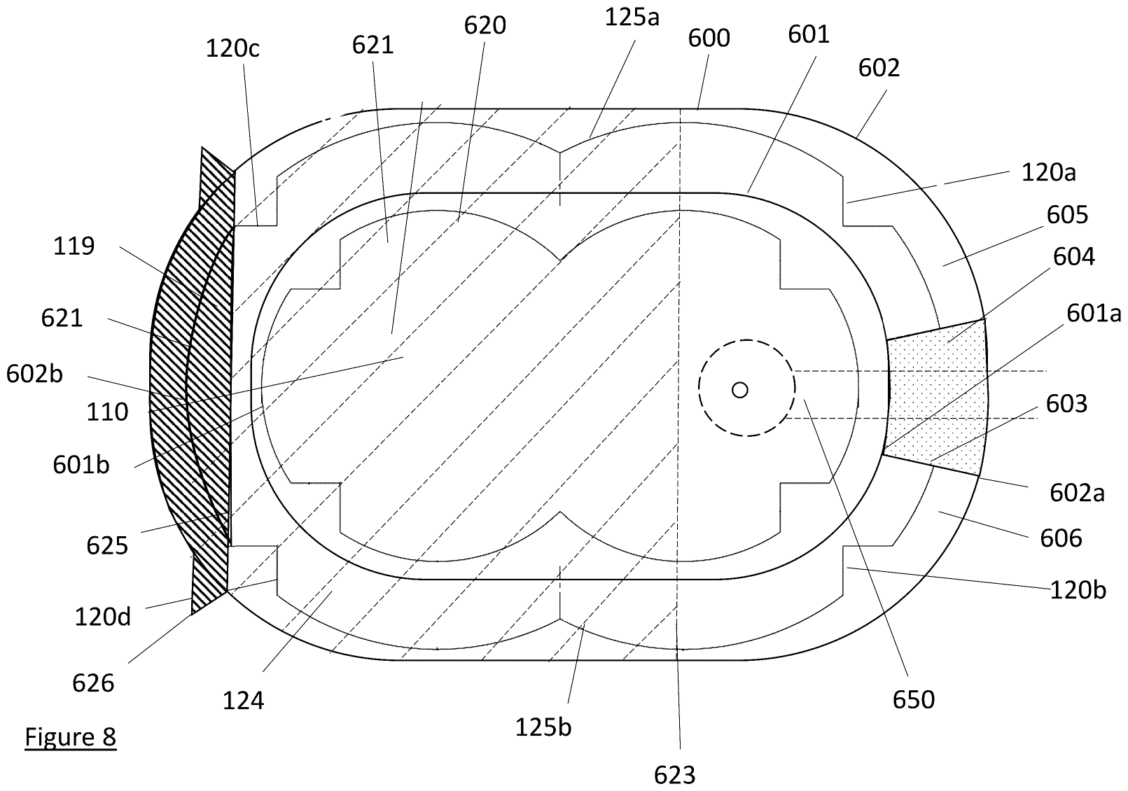

FIG. 8 shows an alternative kit of wound dressing 110 and sealing strip 600. In this kit there is provided one sealing strip 600. The sealing strip 600 is a ring of adhesive tape with adhesive provided on one side. The sealing strip shape is defined by the region between two nestled oblongs with a constant separation between the two, that is an inner edge 601 and an outer edge 602 of the sealing strip both have an oblong shape. The sealing strip 600 has a constant width along its length, the width being greater than that of the border region 124 of the dressing such that in use the sealing strip 600 extends beyond the peripheral edge of the covering layer 119. A slit 603 is provided between a first apex 601a of the inner edge 601 and a first apex 602a of the outer edge 602, additional strip material is provided to allow an overlap 604 of the strip 600 at this slit 603.

The sealing strip 600 is provided on a carrier liner 620 to support the sealing strip before use. The carrier liner 620 is provided in two parts a main liner 621 and a release liner 622. The overall shape of the carrier liner corresponds to the outer edge 602 of the sealing strip about one curved portion and the linear portions of the oblong, where the outer edge 602 has a second curved portion the carrier liner 620 terminates at a carrier edge 623 defined by a line extending perpendicular to the termini of the linear portions of the oblong. As noted above the carrier liner 620 is provided in two parts, the release liner 622 extends from a second apex 602b of the outer edge 602 to a long edge 625 defined by a line perpendicular to, and just beyond, a second apex 601b of the inner edge 601. A release tab 626 is provided on the release liner 622, the release tab 626 is attached to the long edge 625. The carrier liner 621 has a shape defined by the long edge 625 of the release liner, the carrier edge 623 and the outer edges of the sealing strip 602. In addition to the carrier two further release strips 605, 606 are provided on the remaining portions of the sealing strip 600, from the carrier edge 623 to the slot 603.

In use, once the wound dressing 110 has been applied to the wound site the user or HCP places the sealing strip 600 over the border region 124 such that it overlaps and extends beyond the peripheral edge 119 of the covering layer 112. First the release liner 622 is removed by pulling on the release tab 626. The sealing strip can then be aligned with the wound dressing 110 and the exposed adhesive applied to the dressing 110 and wound site. Next the carrier liner 620 can be progressively pealed back to expose further adhesive and the sealing strip adhered about the periphery of the dressing. The overlap allows the sealing strip 600 to be placed under an airway 650 used to connect a source of negative pressure to the wound dressing 110, further release strips are removed one at a time allowing a portion of the sealing strip 600 to be placed under the airway.

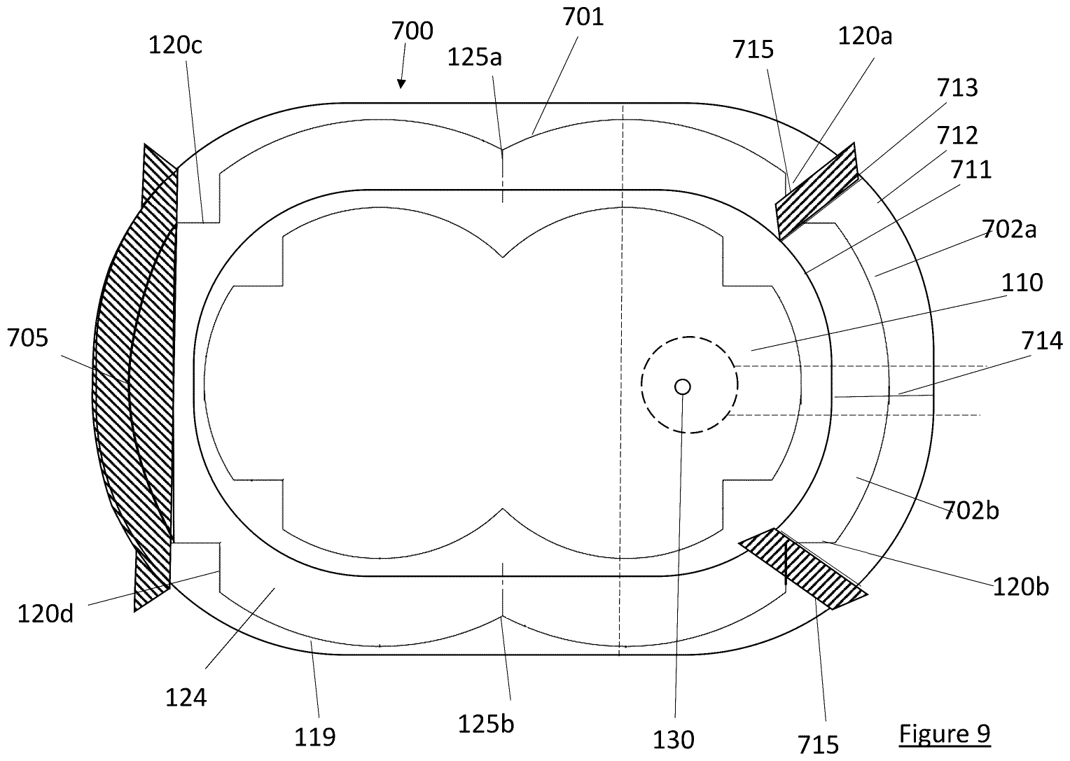

FIG. 9 shows an alternative kit of wound dressing 110 and sealing strip 700. In this kit there is provided a sealing strip 700 comprised of three parts. The sealing strip 700 is shaped such that it matches the shape of the border region 124 of the wound dressing 110, though sized to extend inwards of the peripheral edge of the absorbent pad and outwards of the peripheral edge of the covering layer. A main section 701 of the sealing strip 700 is arranged around the majority of the peripheral edge 119 of the wound dressing 110 from a first covering layer notch 120a on the lobe 114 on which the aperture 130 is provided and the other covering layer notch 120b on the same lobe 114. The sealing strip main section 701 is provided with a release tab 705 at the apex opposite the port 130. The section of the peripheral edge 119 of the wound dressing 110 between the first covering layer notch 120a and the other covering layer notch 120b is covered two further parts of the sealing strip 700 referred to as sealing patches 702a,b. The two sealing patches 702a,b are congruent, one being a reflection of the other, as such only one sealing patch 700a is described in detail. The sealing patch 702a is formed from an annular segment approximately one eighth of a complete annulus having a radially inner edge 711 and a radially outer edge 712, both edges 711, 712 have a common centre point. A first end 713 and a second end 714 of the sealing strip 700a are perpendicular to both the radially inner 711 and outer 712 edges of the strip. The first end 713 is also provided with a release tab 715. As noted above the two shapes of sealing strip in this embodiment are congruent, the only difference being that the release tab 715 is arranged on the opposite end on the second sealing patch 700b.

In use, once the wound dressing 110 has been applied to the wound site the user or HCP places the sealing strips 700 sequentially over the border region 124 such that it overlaps and extends beyond the peripheral edge 119 of the covering layer 112. First the main section 701 of the sealing strip is applied, extending about the majority periphery of the dressing from one covering layer notch 120a to the other covering layer notch 120b in the same lobe 114. Next the two sealing patches 702a,b are applied between the two covering layer notches 120a,b. All three portions of the sealing strip 700 overlap to ensure a good seal.

As will be evident from the above, the number and arrangement of sealing strips can be varied to suit the needs of the user. Using a greater number of sealing strips results in an easier application, but also increases the number of overlapping sections, this can decrease the flexibility of the sealing strips, it also increases the risk of an incomplete seal where to strips meet. Conversely fewer sealing strips are more difficult to accurately place, a particular concern when the dressing is applied by the patient, but increases the quality of the seal.

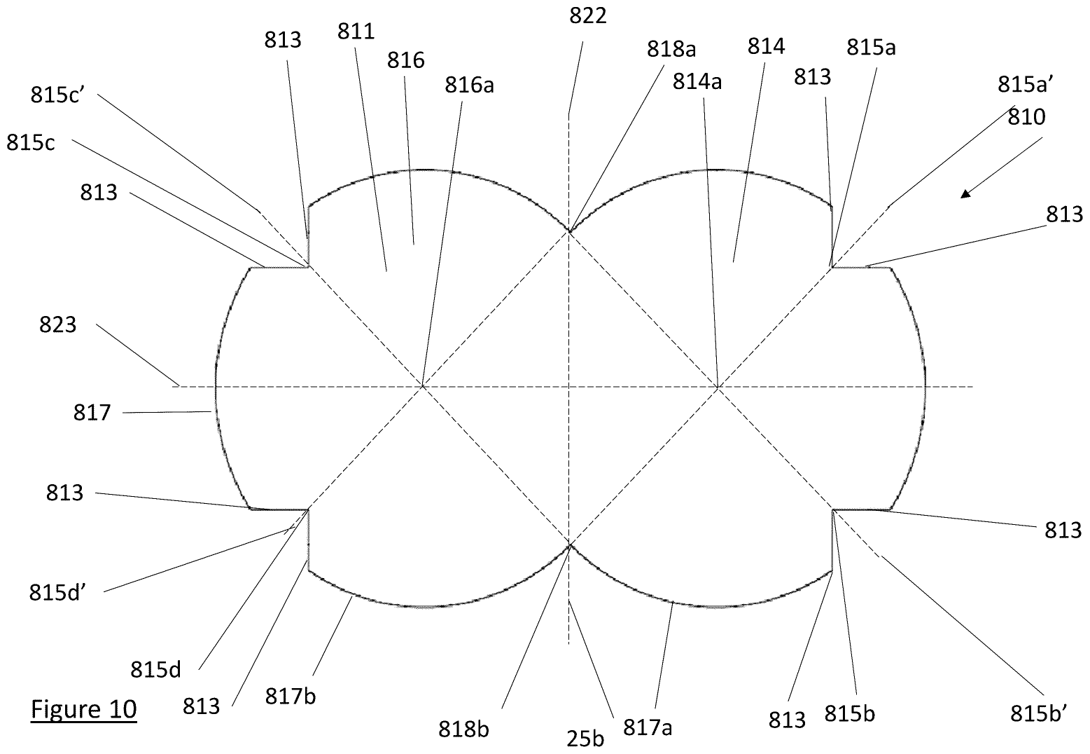

FIG. 10 shows a further wound dressing 800 according to the present disclosure. The most notable difference between this embodiment and those above is that in this embodiment, there is no peripheral adhesive border around the absorbent pad. As such, the periphery of the absorbent pad is the periphery of the dressing, i.e. the outline of the dressing and the outline of the absorbent pad are identical and coterminous. The dressings are considered to be favourable for applying to multiple different sites on the human body with variably or highly contoured aspects which may typically be difficult areas to apply dressing to. These areas may include a shoulder, armpit, elbow, wrist, sacral region, breast, hip and heel, for example.

FIG. 10 shows a wound dressing 810 in plan. The dressing 810 comprises an absorbent pad 811 having a shape comprising two contiguous lobes 814, 816. To aid in the description of the dressing shape, there is shown a centre line 822 which extends along a boundary between the two lobes 814, 816, a second, major axis, line 823 is provided at the midpoint of and perpendicular to the centre line 822, the major axis line 823 extends along a line of symmetry of the dressing 810.

In this embodiment the two lobes 814, 816 are of equal size and have a generally circular shape with each lobe having a respective centre point 814a, 816a, the centre points 14a, 16a being the origin for the circular shape of each lobe 14, 16. The two lobes overlap such that a segment of each defined by an arc and chord with a central angle of 90 degrees is omitted from each lobe. That is, a quarter of the circumference of the circles of each lobe overlaps with the other lobe, such that each lobe extends around 270 degrees of a circle the absorbent pad 811, the dressing thus having a broadly “figure of 8” shape. The peripheral edges of each lobe 817a, 817b meet at two inflection points 818a, 818b, the peripheral edges 817 curving about the respective centre points 814a, 816a until the two peripheral edges 817 meet at an abrupt interface. In other embodiments (which would be asymmetric about the flex line) the two lobes may have different diameters.

In this embodiment each lobe 814, 816 is provided with two notches 815a, 815b, 815c, 815d. To further assist in the description of the dressing shape, FIG. 10 is also provided with notch bisecting lines 815a′ 815b′ 815c′ 815d′, these lines bisect their respective notch 815a, 815b, 815c, 815d. With reference to the first lobe 814, the two notches 815a, 815b are arranged opposing the boundary 89 between the two lobes and symmetrically about the major axis line 823 the angle between the first notch bisecting line 815a′ and the major axis line 823 is 45 degrees about the centre point 814a of the lobe 814. In other embodiments differing numbers of notches may be provided and/or the position of the notches may be varied to various contoured aspects of a wound as will be expanded upon below.

The notches 815 are V-shaped indents in the periphery of the absorbent pad 811, in this embodiment the notches have two straight edges 813 with an internal angle of 90 degrees between them. The notches 815 extend radially inward from the peripheral edge 817 of the absorbent pad 811 one fifth of the way towards the centre point 814a, 816a of their respective lobe 814, 816.

The notches 815 and inflection points 818 provide preferential points from which flex or fold lines in the wound dressing can be formed so as to fit a contoured wound site. A flex line 822 extends between the inflection points 818 along the boundary of the two lobes 814, 816. This flex line 822 allows the wound dressing 810 to be folded to accommodate a ridge or contour in the wound site and thus ensure good adhesion to a non-flat wound site.

Flex lines can also be preferentially formed from one or more of the notches 15, the lines 815a′, 815b′, 815c′, 815d′ bisecting the notch being a notch flex line about which the wound dressing 810 can also be folded.

Due to the alignment of the flex line and notch flex lines, they do not converge at a single point. The notch flex lines 815a′ 815b′ converge in the centre 814a of the first lobe 814 and intersect with the flex line 822 at opposing absorbent pad inflection points 818. Similarly, the notch flex lines 815c′ 815d′ converge in the centre 816a of the second lobe 816 and intersect with the flex line 822 at opposing absorbent pad inflection points 818. The intersections of the flex line and notch flex lines are at angles of 45 degrees.

By arranging the flex line 822 and notch flex lines 815a′, 815b′, 815c′, 815d′ so that they do not all converge at a single point of the dressing the integrity of the dressing 810 is improved. When the dressing 810 is folded the layers of the dressing become non-planar, when more than one fold line intersects the layers can be drawn apart at the intersection point. This can result in separation of the layers and/or loss of contact between the wound site and a wound contact layer which may affect the ability of the dressing to assist in healing. Furthermore, having notch flex lines intersect in their respective lobes 814, 816 allows the lobes to be conformed to the wound site topography independently. The arrangement whereby each notch flex line intersects with the flex line at an inflection point 818, also aids the folding of the dressings about the notch flex lines, and in particular, means that the folding of one lobe along a notch flex line does not affect the shape of the other lobe (since the fold line does not extend into the other lobe).

The provision of notches 815 further improves the conformability of the wound dressing 810 over contours on the wound site. The two edges 813 of an absorbent pad notch 815 can be brought together giving the dressing a shallow conical shape. This can be particularly useful where the dressing 810 is applied to heels, knees, elbows, etc.

As noted above in different embodiments alternative arrangements and alignment of notches may be provided for, allowing the dressing to be conformed to particular wound site contours. For example, each lobe may be provided with four notches allowing the lobe to conform to even more highly contoured wound sites.

Thus, the skilled person will understand that notches can be provided at any point on the peripheral edge of the dressing to provide a terminus for a flex or fold line and thus used to tailor the shape to which the wound dressing 810 can be folded.

To apply the wound dressing 810, the user or health care professional (HCP) can remove the dressing 810 from packaging (not shown) that ensures the sterility of the dressing. The wound dressing 810 can be placed against the wound and the orientation varied to determine the optimal orientation to conform to the wound site contours. As the dressing is applied the user or HCP can shape the dressing by folding it about flex lines between notches 815 and/or by bringing the edges 813 of the notches 815 together. The dressing can be secured to the wound site by adhesive strips, for example the sealing strips described above, bandages or similar means, or a combination thereof.

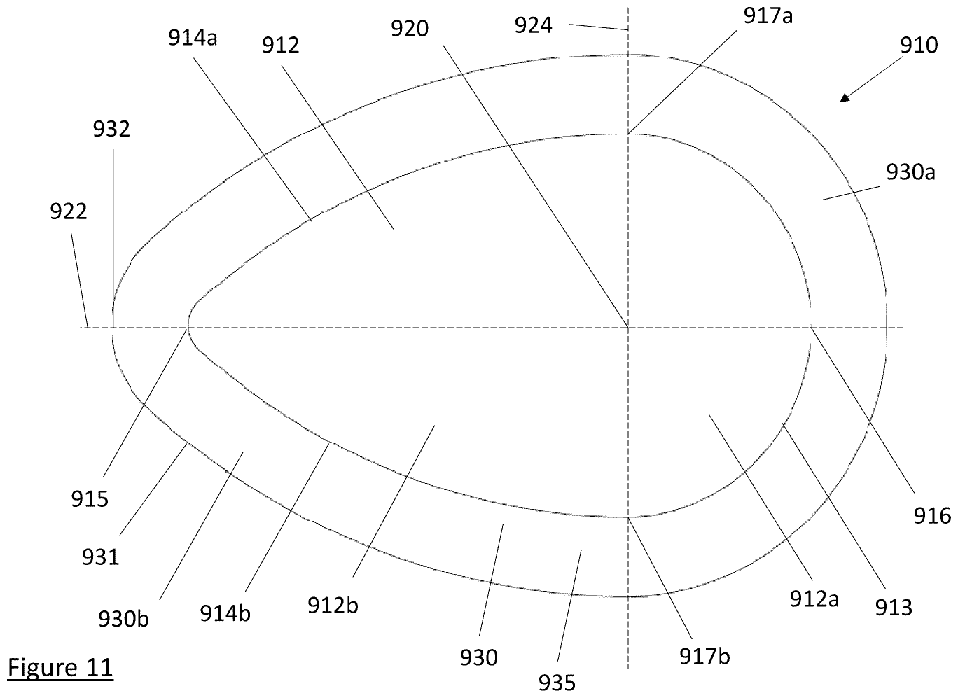

FIG. 11 shows a further wound dressing according to the present disclosure. The dressings are considered to be favourable for attaching or adhering to multiple different sites on the human body.

FIG. 11 shows a wound dressing 910 in plan. The dressing 910 comprises a wound contact layer 912 having a tear-drop shape. To aid the description of the dressing 910 shape, there is shown a centre line 922 which extends centrally along the major axis of the dressing 910 and a transverse midline 924 arranged on the transverse axis perpendicular to the major axis. The length of the dressing 910 is measured along the major axis and the width of the dressing 910, is measured along the transverse axis. The central region of the dressing 910 may generally refer to the area in which a wound would be located when in use, i.e. within a region of the wound contact layer. In some embodiments the central region of the dressing may refer to a circular region centred on the intersection of the centre line 922 and the transverse midline 924 and having a radius less than or equal to the radius of a semi-circular portion of the wound contact layer (as described in detail below). It will be appreciated however that the layers of the wound dressing in the central region 950 does not have a different composition to the remainder of the same layer.

The wound dressing 910 is shaped to conform more readily to different areas of a patient's body which are typically but not necessarily contoured and/or difficult areas to adhere a dressing to. These areas may include a shoulder, armpit, elbow, wrist, sacral region, breast, hip and heel, for example.

As stated above, the wound contact layer 912 has a tear-drop shape, that is it has a semi-circular portion 912a defined by a semi-circular peripheral edge 913 with a tapering portion 912b defined by a pair of taper peripheral edges 914a, b extending away from the flat edge of the semi-circular portion 912a. The two tapering peripheral edges 914a,b are convexly curved and meet at a taper apex 915. The taper apex 915 is rounded (with a radius in this particular embodiment of 10 mm) and is arranged on the major axis 922. Also arranged on the major axis 922 is a circumferential midpoint of the semi-circular portion 916.

The semi-circular peripheral edge 913 and the taper peripheral edges 914a,b meet at a pair of interface points 917a,b; the width wound contact layer is at a maxima between the two interface points 917a,b.

Arranged above the wound contact layer 912 is a covering layer 930, the covering layer 930 is provided with a peripheral edge 931 that extends a constant distance from the peripheral edge 913,914 of the wound contact layer 912 as measured perpendicular to the peripheral edge 913,914 of the wound contact layer. The area between the peripheral edges of the covering layer 931 and wound contact layer 913,914 defines the border region 935 also referred to as a wound-site adhesive layer 935 with a contact adhesive provided on the first surface thereof.

As the covering layer 930 extends an equal distance about the wound contact layer 912, the covering layer also has a semi-circular portion 930a and a tapered portion 930b which share a boundary along the transverse midline 924. The tapered portion 930b terminates at an apex 932, like on the wound contact layer the tapered portion apex 932 is arranged on the centreline 922. A circumferential midpoint 933 of the semi-circular portion 930a is also arranged on the centreline 922. As with the wound contact layer 912, the tapered portion apex 932 is also rounded. As the radius of the rounded apex is greater than that of the rounded apex of the wound contact layer the covering layer 930 has a more ovate shape. The tapered portion apex 932 is rounded to reduce instances where the apex peels away from the wound site as can occur where the taper is not rounded.

In this embodiment the wound dressing 910 has a length of 270 mm, and a width of 190 mm, the radius of the semi-circular portion is 95 mm and the length of the taper is 175 mm and the rounded apex of the tapered portion has a radius of 35 mm. The wound contact layer has a length of 200 mm and a width of 120 mm, the radius of the semi-circular portion is 60 mm and the tapered portion has a length of 140 mm and the rounded apex of the tapered portion has a radius of 10 mm. Accordingly, the border region 935 has a constant width of 35 mm.

Of course, the aforementioned teachings could be applied to dressings of other sizes. For example, the dressing could be uniformly scaled down for use with smaller wound sites, or uniformly scaled up for larger wound sites. Equally, the dressing may be non-uniformly scaled, thereby providing a more elongate dressing or a wider dressing as required to suit a particular wound site whilst still retaining the overall tear-drop shape. Likewise, the width of the border region could be varied, for example from between 10 mm and 50 mm with a wider border region providing greater adhesion to the wound site.

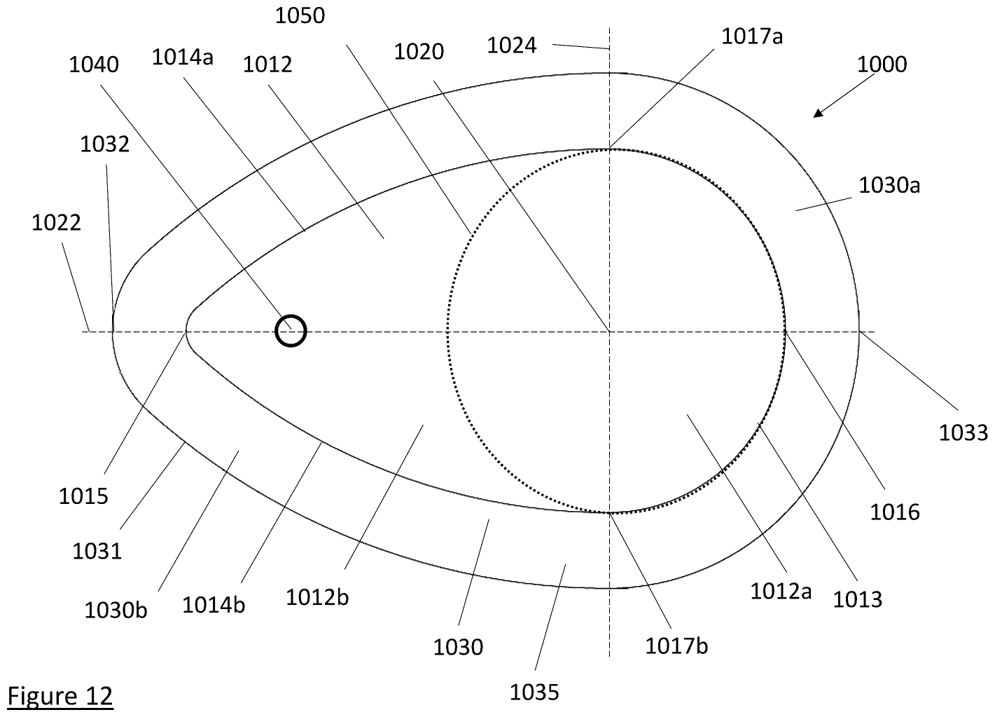

FIG. 12 shows a further wound dressing 1000 according to the present disclosure. In this embodiment the wound dressing 1000 is configured for use with a source on non-atmospheric pressure (not shown), for example for use in Negative Pressure Wound Therapy (NPWT). The tear-drop shape of the wound dressing 1000 is particularly suited to applications such as NPWT as it maximises the area for applying the treatment to the wound whilst minimising the amount of additional dressing required to accommodate a port for the non-atmospheric pressure source as will be expanded upon below.

FIG. 12 shows the wound dressing 1000 in plan. The dressing 1000 comprises a wound contact layer 1012 having a tear-drop shape. To aid the description of the dressing shape, there is shown a centre line 1022 which extends centrally along the major axis of the dressing 1000 and a transverse midline 1024 arranged on the transverse axis perpendicular to the major axis. The length of the dressing 1000 is measured along the major axis and the width of the dressing 1000 is measured along the transverse axis. The central region 1050 of the dressing 1000 may generally refer to the area in which a wound would be located when in use, i.e. within a region of the wound contact layer. In some embodiments the central region of the dressing may refer to a circular region centred on the intersection of the centre line 1022 and the transverse midline 1024 and having a radius less than or equal to the radius of a semi-circular portion of the wound contact layer (as described in detail below). It will be appreciated however that the layers of the wound dressing in the central region 1050 does not have a different composition to the remainder of the same layer.

The wound dressing 1000 is shaped to conform more readily to different areas of a patient's body which are typically but not necessarily contoured and/or difficult areas to adhere a dressing to. These areas may include a shoulder, armpit, elbow, wrist, sacral region, breast, hip and heel, for example.

As stated above, the wound contact layer 1012 has a tear-drop shape, that is it has a semi-circular portion 1012a defined by a semi-circular peripheral edge 1013 with a tapering portion 1012b defined by a pair of taper peripheral edges 1014a,b extending away from the flat edge of the semi-circular portion 1012a. The two taper peripheral edges 1014a, b are convexly curved and meet at a taper apex 1015. The taper apex 1015 is rounded with a radius of 10 mm and is arranged on the major axis 1022. Also arranged on the major axis 1022 is a circumferential midpoint of the semi-circular portion 1016.

The semi-circular peripheral edge 1013 and the taper peripheral edges 1014a,b meet at a pair of interface points 1017a,b, the width wound contact layer is at a maxima between the two interface points 1017a,b.

Arranged above the wound contact layer 1012 is a covering layer 1030, the covering layer 1030 is provided with a peripheral edge 1031 that extends a constant distance from the peripheral edge 1013,1014 of the wound contact layer 1012 as measured perpendicular to the peripheral edge 1013,1014 of the wound contact layer. The area between the peripheral edges of the covering layer 1031 and wound contact layer 1013,1014 defines the border region 1035 also referred to as a wound-site adhesive layer 1035 with a contact adhesive provided on the first surface thereof. The peripheral edge of the covering layer 1031 also defines the peripheral edge of the wound dressing.

The covering layer 1030 of the dressing is provided as a bacterial and viral barrier layer which preferably resists the ingress of liquid and air but allows moisture vapour transmission. In this way the cover layer enhances the overall fluid handling capacity of the dressing by allowing for the escape of moisture vapour through the cover while enabling the application of pressure (either positive or negative) to the wound. The outer cover layer is for instance a layer having a MVTR of at least 3,000 g m⁻² per 24 hours or, more preferably, in the range of from 10,000 gm⁻² to 50,000 g m⁻² per 24 hours measured by the method described in BS EN 13726-2 2002 “Test methods for primary wound dressings Part 2 Moisture vapour transmission rate of permeable film dressings”. The cover layer may be in the form of a film of polyurethane, for example Inspire 2350 manufactured by Coveris.

The covering layer 1030 and wound define a wound dressing cavity in which the remaining layers of the dressing 1000 are provided.

As the covering layer 1030 extends an equal distance about the wound contact layer 1012, the wound contact layer also has a semi-circular portion 1030a and a tapered portion 1030b which share a boundary along the transverse midline 1024. The tapered portion 1030b terminates at an apex 1032, like on the wound contact layer the tapered portion apex 1032 is arranged on the centreline 1022. The tapered portion apex 1032 is rounded to reduce instances where the apex was to peel away from the wound site as can occur where the taper is not rounded. As the radius of the rounded apex 1032 of the covering layer 1030 is greater than that of the rounded apex 1015 of the wound contact later 1012 the covering layer 1030 has a move ovate shape. A circumferential midpoint 1033 of the semi-circular portion 1030a is also arranged on the centreline 1022.

The covering layer 1030 is also provided with an aperture 1040, the aperture is arranged in the tapered portion 1012b. The aperture is positioned on the centreline 1022 approximately 25% of the distance from the peripheral edge of the wound contact layer to the 1012 to the centre 1020. As such the aperture is arranged outside of the central region 1050. The aperture provides fluid communication between the wound dressing cavity and an environment external to the dressing. In some embodiments the aperture is simply an opening in the covering layer 1030, in other embodiments the aperture is provided with a port (not shown), the port is configured to connect to a length of tubing supplying the non-atmospheric pressure, in still further embodiments (not shown) an airway is connected to the aperture, the airway being configured to connect to a length of tubing supplying non-atmospheric pressure.

Providing the aperture 1040 on the tapered portion 1012b is advantageous because it is beneficial to the user that the connection to the non-atmospheric pressure (e.g. port or airway) is not arranged above the wound site. In instances where the port is provided above the wound site an external application of pressure onto the dressing, for example where the user lies on it or rests against it, applies greater pressure at the port due to it raised nature. Placing the aperture 1040 on the tapered portion 1012b allows the central region 1050 to be applied over the wound site and the tapered portion to be laterally offset from the would site, the tear-drop shape limits the amount of material required to provide this lateral offset of the aperture 1040.

In this embodiment the wound dressing 1000 has a length of 250 mm, and a width of 170 mm, the radius of the semi-circular portion is 85 mm and the length of the taper is 165 mm and the rounded apex of the tapered portion has a radius of 35 mm. The wound contact layer has a length of 200 mm and a width of 120 mm, the radius of the semi-circular portion is 60 mm and the tapered portion has a length of 140 mm and the rounded apex of the tapered portion has a radius of 10 mm. Accordingly, the border region 1035 has a constant width of 25 mm.

Of course, the aforementioned teachings could be applied to dressings of other sizes. For example, the dressing could be uniformly scaled down for use with smaller wound sites, or uniformly scaled up for larger wound sites. Equally, the dressing may be non-uniformly scaled, thereby providing a more elongate dressing or a wider dressing as required to suit a particular wound site whilst still retaining the overall tear-drop shape. Likewise, the width of the border region could be varied, for example from between 10 mm and 50 mm with a wider border region providing greater adhesion to the wound site and improve the seal when a source of non-atmospheric pressure is provided.

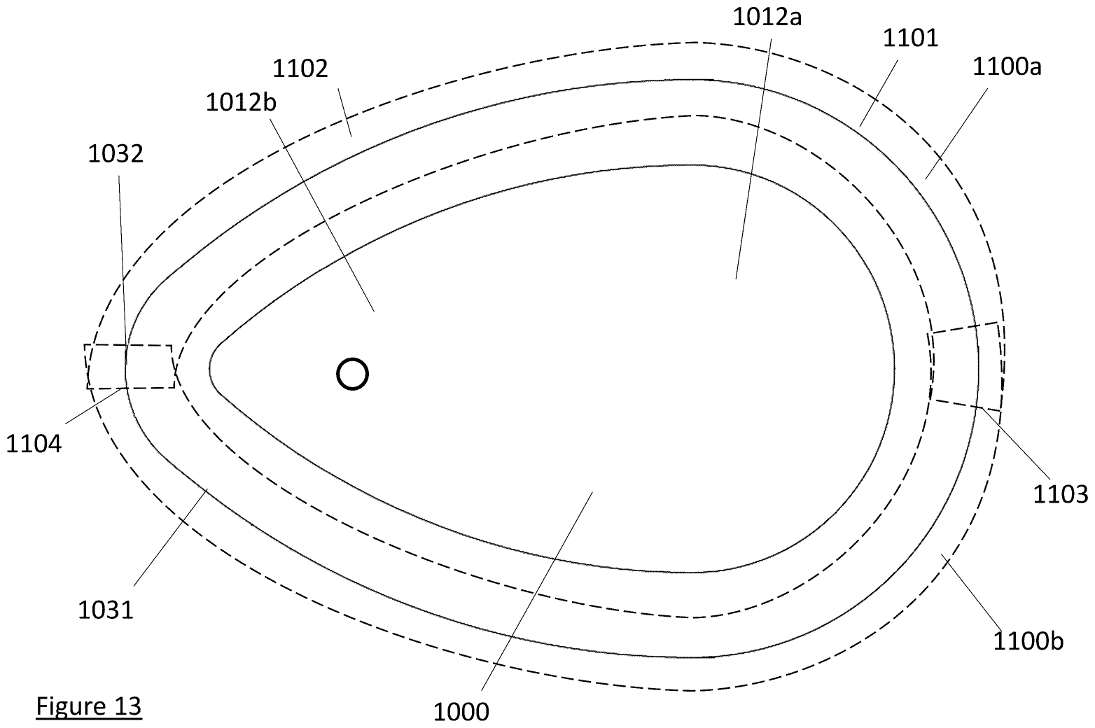

In some embodiments the wound dressing is supplied as part of a kit as shown in FIG. 13. The kit contains one or more sealing strips 1100. In this embodiment there are two sealing strips 1100a,b. The sealing strips 1100a,b are segments of adhesive tape having an adhesive on one side. The sealing strips 1100a,b are provided to ensure that the dressing maintains an airtight seal when a source of non-atmospheric pressure is applied. As such the sealing strips overlap the peripheral edge 1031 of the wound dressing.

The two strips 1100a,b are geometrically similar being reflections of one another. As such only one sealing strip 1100a will be described in detail.

As noted above the sealing strip 1100a overlap the peripheral edge 1031 of the wound dressing and therefore have a profile which follows this periphery. The shape of the sealing strip 1100a has two portions 1101,1102 corresponding to the two portions of the wound dressing 1022a,b. The first portion has an arc profile covering approximately 100 degrees of a circle, a first end 1103 of this arc portion 1101 forms an end of the sealing strip, the second end of the arc portion is integrally formed with the second portion 1102 of the sealing strip 1100a. The second portion 1102 has a curved profile that curves in the same sense as the arc 1101 such that the whole sealing strip 1100a is a concave curve the distal end of which forms the second end 1104 of the strip.

In use the wound dressing 1000 is applied to the wound site using the wound-site adhesive layer to secure it in place. The source of non-atmospheric pressure is then attached to the dressing via the aperture 1040. The sealing strips 1100 are then applied to the perimeter of the dressing 1000 to ensure that there are no leaks, the ends of the strips overlapping at the apexes 1032,1033.

In further embodiments the wound dressing 1000 is supplied as part of a Negative Pressure Wound Therapy system 1200 as shown in FIG. 14. The kit comprises one or more wound dressings 1000 configured for used with NPWT, and a pump 1210 to supply a source of negative pressure. The kit may also comprise one or more lengths of pressure tubing 1220 that can be used to connect the wound dressing 1000 to the pump 1210. In some embodiments the system 1200 also includes one or more sealing strips 1100 as described above.

In use the wound dressing 1000 is applied to the wound site using such that the central region 1050 is arranged over the wound using the wound-site adhesive layer to secure it in place. The pump 1210 is then connected to a first end of the pressure tubing 1220 and a second end of the pressure tubing 1220 is attached to the dressing via the aperture 1040 (optionally via an airway, not shown), thus the wound dressing cavity is in fluid communication with the pump. In some embodiments sealing strips 1100 are then applied to the perimeter of the dressing 1000 to ensure that there are no leaks, the ends of the strips overlapping at the apexes 1032,1033.

The wound dressings of the present disclosure are shaped to lend themselves to improved patient comfort during NPWT.

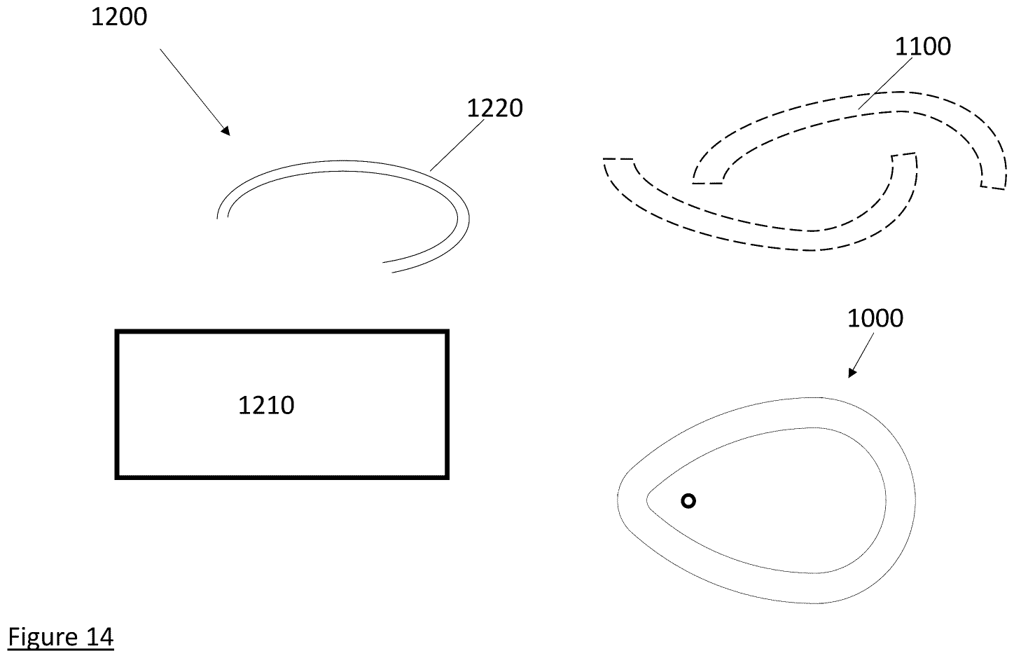

FIG. 15 shows a wound dressing according to the present disclosure. The dressings are considered to be favourable for user comfort when applied to a wound site during NPWT.

FIG. 15 shows a wound dressing 1310 in plan. The dressing 1310 comprises a wound contact layer 1312 having an oval shaped outline, which may be an elliptical shape, as illustrated. To aid the description of the dressing 1310 shape, there is shown a centre line 1322 which extends centrally along the major axis of the wound contact layer 1310 and a transverse midline 1324 arranged on the minor axis of the wound contact layer 1312 perpendicular to the major axis. The length of the dressing 1310 is measured along the major axis and the width of the dressing 1310, is measured along the minor axis. A central region 1350 of the dressing 1310 may generally refer to the area in which a wound would be located when in use, i.e. within a region of the wound contact layer. In some embodiments the central region of the dressing may refer to a circular region centred on the intersection of the centre line 1322 and the transverse midline 1324 (referred to as the centre point 1320) and having a diameter equal to the width of the wound contact layer (as described below). It will be appreciated however that the layers of the wound dressing in the central region 1350 need not have a different composition to the remainder of the same layer.

As stated above, the wound contact layer 1312 has an oval shape, it is bounded by a peripheral edge 1313, being generally round with a length greater than its width.

Arranged above the wound contact layer 1312 is a covering layer 1330, the covering layer 1330 is provided with a peripheral edge 1331 that may extend a constant distance from the peripheral edge 1313 of the wound contact layer 1312 as measured perpendicular to the peripheral edge 1313 of the wound contact layer. The area between the peripheral edges of the covering layer 1331 and wound contact layer 1313 may define a border region 1335 which may also be referred to as a wound-site adhesive layer 1335 in instances where a contact adhesive provided on the first surface of the border region 1335. The peripheral edge 1331 of the covering layer 1330 may also form the peripheral edge of the wound dressing 1310.

In instances where the covering layer 1330 extends an equal distance about the wound contact layer 1312, the covering layer 1330 also has an oval shaped outline, the major and minor axes of which are superimposed with the major and minor axes of the wound contact later 1312. In other instances, the ovals may have different shapes and/or the major and/or minor axes of the covering layer may be laterally offset from the major and/or minor axes of the wound contact layer 1312. The oval shape intersects the major axis at two major axis apexes 1332a,b and intersects the minor axis at two minor axis apexes 1333a,b

The covering layer 1330 and wound may define a wound dressing cavity in which the remaining layers of the dressing 1310 are provided.

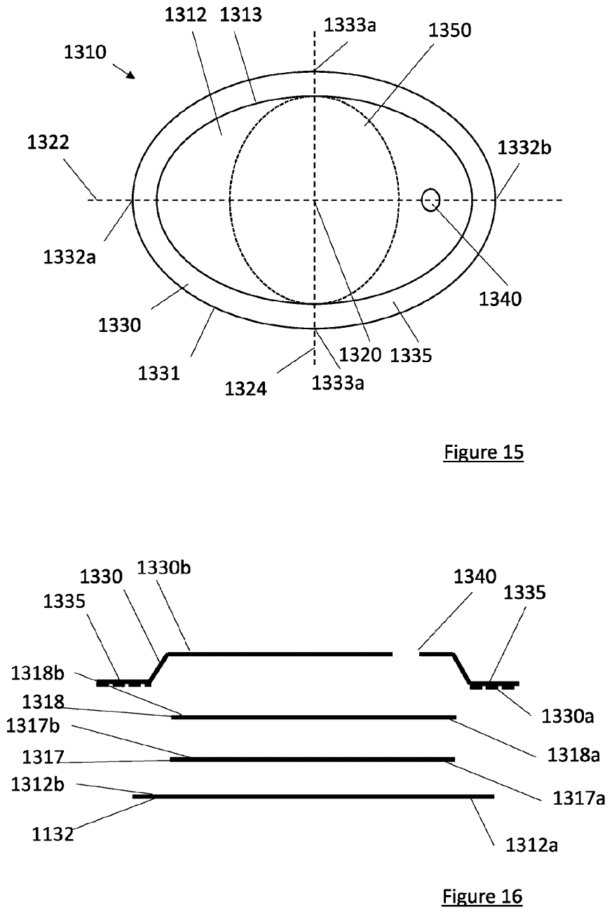

FIG. 16 shows an exploded view of the layers of the wound dressing 1310. In this embodiment there is provided a transmission layer 1317 arranged adjacent to the wound contact layer 1312 and a super absorbent layer 1318 arranged between the transmission layer 1317 and the covering layer 1330. Together the wound contact layer 1312, transmission layer 1317 and super absorbent layer 1318 form an absorbent structure arranged in the wound dressing cavity between the wound and the covering layer 1330.

The covering layer 1330 may comprise a first surface 1330a and a second surface 1330b. In use, the first surface 1330a may be inwardly facing toward the patient's body, and the second surface 1330b may be outwardly facing away from the patient's body.

The absorbent structure 1312, 1317, 1318 may comprise a first surface 1312a and a second surface 1318b. The first surface 1312a of the absorbent structure 1312, 1317, 1318 may be adjacent, and in contact with, the wound. The peripheral edge of the second surface 1318b of the absorbent structure 1312, 1317, 1318 may be adjacent, and in contact with, the first surface 1330a of the backing layer 1330.

The wound contact layer 1312 may comprise a first surface 1312a and a second surface 1312b. In use, the first surface 1312a may be inwardly facing toward the patient's body, and the second surface 1312b may be outwardly facing away from the patient's body. The first surface 1312a of the wound contact layer 1312 may contact the wound when the wound dressing 1310 is adhered to the skin adjacent to the wound (i.e., when in use). The second surface 1312b of the wound contact layer 1312 may be adjacent, and in contact with, a first surface 1317a of the transmission layer 1317 or a first surface 1318a of the superabsorbent layer 1318. In the described embodiment, the second surface 1312b of the wound contact layer 1312 is adjacent, and in contact with, the first surface 1317a of the transmission layer 1317.

The transmission layer 1317 may comprise a first surface 1317a and a second surface 1317b. In use, the first surface 1317a may be inwardly facing toward the patient's body, and the second surface 1317b may be outwardly facing away from the patient's body. The first surface 1317a of the transmission layer 1317 may be adjacent, and in contact with, the second surface 1312b of the wound contact layer 1312 or a second surface 1318b of the superabsorbent layer 1318. The second surface 1317b of the transmission layer 1317 may be adjacent, and in contact with, a first surface 1318a of the superabsorbent layer 1318 or the first surface 1330a of the backing layer 1330. In the described embodiment, the first surface 1317a of the transmission layer 1317 is adjacent, and in contact with, the second surface 1312b of the wound contact layer 1312, and the second surface 1317b of the transmission layer 1317 is adjacent, and in contact with, the first surface 1318a of the superabsorbent layer 1318.

The superabsorbent layer 1318 may comprise a first surface 1318a and a second surface 1318b. In use, the first surface 1318a may be inwardly facing toward the patient's body, and the second surface 1318b may be outwardly facing away from the patient's body. The first surface 1318a of the superabsorbent layer 1318 may be adjacent, and in contact with, the second surface 1312b of the wound contact layer 1312 or the second surface 1317b of the transmission layer 1317. The second surface 1318b of the superabsorbent layer 1318 may be adjacent, and in contact with, the first surface 1317a of the transmission layer 1317 or the first surface 1330a of the backing layer 1330. In the described embodiment, the first surface 1318a of the superabsorbent layer 1318 is adjacent, and in contact with, the second surface 1317b of the transmission layer 1317, and the second surface 1318b of the superabsorbent layer 1318 is adjacent, and in contact with, the first surface 1330a of the backing layer 1330.

The wound contact layer 1312 may radially overlap the transmission layer 1317 and the superabsorbent layer 1318 and extend beyond the periphery thereof. The second surface 1312b of the wound contact layer may in the region extending beyond the transmission layer 1317 and super absorbent layer 1318 be joined to the first surface 1330a of the covering layer 1330 by heat lamination, adhesive, welding or stitching.

The wound contact layer 1312 may radially beyond the periphery of the transmission layer 1317 and superabsorbent layer 1318 by about 12.5 mm.

Beneficially, by providing a wound contact layer 1312 that extends beyond the periphery of the transmission layer 1317 and superabsorbent layer 1318 and is joined to the first surface 1330a of the covering layer 1330 the remaining layer can be retained within the wound dressing.

At least two of the wound contact layer 1312, the transmission layer 1317 and the superabsorbent layer 1318 may be laminated together. In the described embodiment, each of the wound contact layer 1312, the transmission layer 1317 and the superabsorbent layer 1318 are laminated together. Advantageously, this means that the absorbent structure of the present invention is manufactured without requiring any relatively rigid dressing and binding components. Thus, the present invention provides an absorbent structure which is flexible, therefore benefits user comfort, and which is cheaper to manufacture than absorbent structures and, therefore negative pressure wound dressings, of the prior art. Moreover, lamination does not require any complex equipment or manufacturing techniques. Thus, the present invention is simple to manufacture.

The covering layer 1330 is also provided with an aperture 1340, the aperture 1340 may be arranged outside of the central region 1350. The aperture may be positioned on the centreline 1322. The aperture 1340 may be approximately 25% of the distance from the peripheral edge 1313 of the wound contact layer to the 1312 to the centre 1320. As such the aperture 1340 is arranged outside of the central region 1350. The aperture 1340 provides fluid communication between the wound dressing cavity and an environment external to the dressing. In some embodiments the aperture 1340 is simply an opening in the covering layer 1330, for connection to an airway or port, in other embodiments the aperture is provided with an airway 1341 or port. The airway 341 may be configured to connect to pressure tubing supplying the non-atmospheric pressure.

Providing the aperture outside the central region 1350 is advantageous because it is beneficial to the user that the connection to the non-atmospheric pressure (e.g. airway or port) is not arranged above the wound site. In instances where the airway 1341 or port is provided above the wound site an external application of pressure onto the dressing, for example where the user lies on it or rests against it, applies greater pressure at the port due to it raised nature. Placing the aperture 1340 outside the central region 1350 allows the central region 1350 to be applied over the wound site and the port 1341 to be laterally offset from the wound site, e.g. above the periwound. Similarly, as shown in FIG. 18 the airway extends away from the central region 1350. As with the port this avoids a further source of external application of pressure onto the dressing above the wound site when the user lies or it rests against it.

The wound dressing 1310 may have a length of 240 mm and may have a width of 190 mm. The wound contact layer may have a length of 170 mm and may have a width of 120 mm. The border region 1335 may have a constant width of at least 30 mm, preferably 35 mm.

Of course, the aforementioned teachings could be applied to dressings of other sizes. For example, the dressing could be uniformly scaled down for use with smaller wound sites, or uniformly scaled up for larger wound sites. Equally, the dressing may be non-uniformly scaled, thereby providing a more elongate dressing or a wider dressing as required to suit a particular wound site whilst still retaining the overall oval shape. Likewise, the width of the border region could be varied, for example from between 10 mm and 50 mm with a wider border region providing greater adhesion to the wound site.

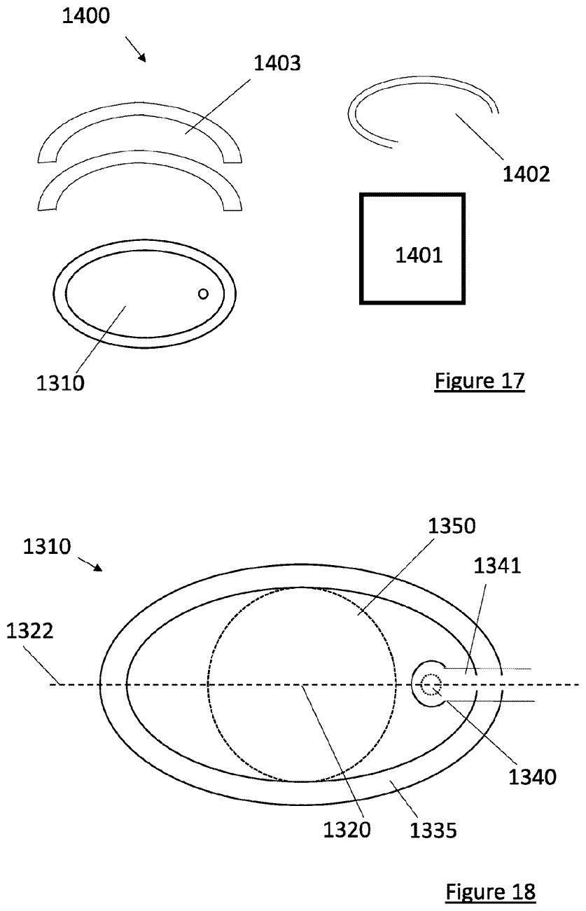

In further embodiments the wound dressing 1310 is supplied as part of a kit for providing a Negative Pressure Wound Therapy system 1400 as shown in FIG. 17. The kit comprises one or more wound dressings 1310 configured for use with NPWT (or positive pressure gradient therapy) and a pump 1401 to supply a source of negative (or positive) pressure. The kit also comprises one or, optionally, more lengths of pressure tubing 1402 that can be used to connect the wound dressing 1310 to the pump 1401. In some embodiments the system 1400 also includes one or more sealing strips 1403. In this embodiment there are two sealing strips 1403, but it is envisaged more or less may be provided. Fewer sealing strips improve the seal but increase the difficulty of application due to their increased size. More sealing strips are easier to apply but introduce the risk of gaps in the seal as there are more joins between strips. The sealing strips 1403 may be segments of adhesive tape having an adhesive on one side. The sealing strips 1403 may be provided to ensure that the dressing 1300 maintains an airtight seal when a source of non-atmospheric pressure is applied. As such the sealing strips may overlap the peripheral edge 1331 of the wound dressing.

In this embodiment the two strips 1403 are identical, as such only one sealing strip 1403 will be described in detail.

As noted above the sealing strips 1403 overlap the peripheral edge 1331 of the wound dressing, extending between (and slightly over) the major axis apexes 1332 and therefore have a profile which follows this periphery. As will be appreciated by those skilled in the art, where a greater number of sealing strips are provided, the geometry of the wound dressing periphery may necessitate that sealing strips with different shapes are provided in the same kit.

In use the wound dressing 1310 may be applied to the wound site such that the central region 1350 is arranged over the wound using the wound-site adhesive layer to secure it in place. The pump 1401 may then connected to a first end of the pressure tubing 1402 and a second end of the pressure tubing 1402 is attached to the dressing 1310 via the aperture 1340 via the airway 1341 (or alternatively a port), thus the wound dressing cavity is in fluid communication with the pump 1401. In some embodiments sealing strips 1403 are then applied to the perimeter of the dressing 1300 to ensure that there are no leaks, the ends of the strips overlapping at the apexes 1432,1433.

The one or more embodiments are described above by way of example only. Many variations are possible without departing from the scope of protection afforded by the appended claims.

In particular the exemplary embodiments describe a dressing of one size, it will be appreciated that the teaching could readily be applied to larger or smaller dressings suited to different wound types, sizes and locations.

1. A wound dressing having a periphery having an outline,
the wound dressing comprising an absorbent pad having an outline,
wherein the outline of the periphery and the outline of the absorbent pad have the same shape,
the shape comprising two lobes, the boundary between the two lobes defining a flex line,
at least one lobe comprising a least one notch.

2. A wound dressing according to claim 1 wherein a line bisecting the notch is at an acute angle to the flex line.

3. A wound dressing according to claim 2 wherein the acute angle is between 30 and 60 degrees.

4. A wound dressing according to any preceding claim comprising at least two notches.

5. A wound dressing according to claim 4 wherein each notch is bisected by a line and each bisecting line is at an acute angle to the flex line.

6. A wound dressing according to any preceding claim wherein the at least one notch comprises two straight edges.

7. A wound dressing according to claim 6 wherein the straight edges are arranged at 90 degrees to one another.

8. A wound dressing according to any preceding claim wherein the wound dressing comprises at least two layers, the absorbent pad and a wound-site adhesive layer for adhering the wound dressing to the wound.

9. A wound dressing according to claim 8 wherein a plurality of notches are provided on both the absorbent pad and the wound-site adhesive layer.

10. A wound dressing according to claim 9 wherein a notch on the absorbent pad is aligned with a notch on the wound-site adhesive layer to form a notch pair.

11. A wound dressing according to claim 8 or 9 wherein each notch on the absorbent pad is aligned with a notch on the wound-site adhesive layer to form a notch pair.

12. A wound dressing according to claim 10 or 11 wherein both notches in the notch pair have the same profile.

13. A wound dressing according to any of claims 10 to 12 wherein both notches in the notch pair have the same bisecting line.

14. A wound dressing according to any preceding claims wherein the wound dressing is symmetrical about the flex line.

15. A wound dressing according to any preceding claim wherein the wound dressing has two lobes.

16. A would dressing according to any preceding claim wherein each lobe has a part-circular outline.

17. A wound dressing according to claim 15 or 16 wherein the shape of the two lobes comprises overlapping circles.

18. A wound dressing according to any of claims claim 15 to 17 when directly or indirectly dependent upon claim 2 wherein the line bisecting the notch intersects a centre of the corresponding lobe.

19. A wound dressing according to any preceding claim wherein each lobe comprises at least one notch.

20. A wound dressing according to any preceding claim wherein at least one lobe comprises at least two notches.

21. A wound dressing according to any preceding claim wherein each lobe has two notches.

22. A wound dressing according to claims 20 or 21 wherein lines bisecting each notch intersect at a point other than on the flex line.

23. A wound dressing according to any of claims 20 to 22 wherein the lines bisecting each notch intersect at the centre of their corresponding lobe.

24. A wound dressing according to claim 8 wherein perforations are provided in the wound-site adhesive layer along the flex line, between a periphery of the wound-site adhesive layer and a periphery of the absorbent pad.

25. A wound dressing according to any preceding claim, wherein the wound dressing is provided in packaging.

26. A wound dressing according to any preceding claim, wherein the wound dressing is provided in a kit for use in a pressure gradient wound therapy system, the kit further comprising a least one sealing strip, the sealing strip comprising an adhesive backed tape and shaped to overlap at least a portion of the perimeter of the wound dressing.

27. A wound dressing according to claim 26 where in the kit comprises four sealing strips, the sealing strips having a half annular shape.

28. A wound dressing comprising at least one layer having a tear-drop shaped outline, the tear-drop shape comprising a semi-circular portion and a tapered portion, wherein the tapered portion terminates in a rounded edge.

29. A wound dressing according to claim 28 comprising a wound contact layer, wherein the wound contact layer has a tear-drop shape.

30. A wound dressing according to claim 28 or 29 comprising a covering layer, wherein the covering layer has a tear-drop shape.

31. A wound dressing according to claim 28 or 29 wherein the wound dressing has an ovate outline.

32. A wound dressing according to any of claims 28 to 31 wherein the wound dressing comprises at least two layers, a wound contact layer and a covering layer.

33. A wound dressing according to claim 32 wherein the covering layer extends an equal distance beyond a perimeter of the wound contact layer about the entire perimeter.

34. A wound dressing according to claim 32 or 33 wherein the wound contact later has a tear-drop shaped outline and the covering layer has an ovate outline.

35. A wound dressing according to any of claims 32 to 34 wherein the wound dressing further comprises an absorbency layer and a pressure distribution layer.

36. A wound dressing according to claim 35 wherein the wound contact layer, absorbency layer and pressure distribution layer have the same shape.

37. A wound dressing according to any of claims 32 to 36 wherein a region of the covering layer extending between a perimeter of the wound contact layer and a perimeter of the covering layer is provided with an adhesive.

38. A wound dressing according to any of claims 28 to 37 further comprising a major axis, the major axis extending parallel to the taper of the tapered portion, wherein the wound dressing is symmetrical about the major axis.

39. A wound dressing according to any of claims 28 to 38 wherein the wound dressing is configured for use with a source of non-atmospheric pressure, the wound dressing further comprising:
a wound contact layer having a first surface for contacting the wound and an opposing second surface; and
a covering layer having a first surface facing the wound contact layer and defining a wound dressing cavity and an opposing second surface wherein the wound contact layer is arranged within the cavity;
the covering layer further comprising an aperture configured to provide fluid communication between the wound dressing cavity and a source of non-atmospheric pressure.

40. A wound dressing according to claim 39 wherein the wound contact layer has a tear-drop shape comprising a semi-circular portion and a tapered portion.

41. A wound dressing according to claim 40 when indirectly dependent upon claim 38 wherein the wound dressing further comprises a transverse axis arranged along a boundary between the semi-circular portion and tapered portion of the wound contact later, perpendicular to the major axis, wherein the two axes intersect at a centre point, the wound dressing further comprising a circular central region centred on the centre point and having a radius less than or equal to the radius of the semi-circular portion of the wound contact layer.

42. A wound dressing according to any of claims 39 to 41 wherein the aperture is arranged on the tapered portion of the dressing.

43. A wound dressing according to claims 41 or 42 wherein the aperture is arranged outside of the central region.

44. A wound dressing according to any of claims 39 to 43 wherein the aperture is provided with a port.

45. A wound dressing according to any of claims 39 to 44 wherein the wound dressing is provided as part of a kit, the kit further comprising one or more sealing strips.

46. A wound dressing according to claim 45 wherein the kit comprises two sealing strips, wherein each sealing strip is shaped to overlap the perimeter of the wound dressing between an apex of the semi-circular portion and an apex of the tapered portion, the two sealing strips overlapping at the apexes.

47. A pressure gradient wound therapy system, the system comprising:
a wound dressing comprising at least one layer having a tear-drop shaped outline;
a source of non-atmospheric pressure; and
a first length of pressure tubing.

48. A pressure gradient wound therapy system according to claim 47 wherein the system is a negative pressure wound therapy system, the source of non-atmospheric pressure being a source of negative pressure.

49. A pressure gradient wound therapy system according to claim 47 or 48 wherein the wound dressing is a wound dressing according to any of claims 28 to 44.

50. A method of applying a wound dressing comprising at least one layer having a tear-drop shaped outline to a wound site.

51. A method according to claim 50 wherein the wound dressing is a wound dressing according to any of claims 28 to 44.

52. A method according to claim 50 or 51 further comprising placing a central region of the wound dressing over the wound site.

53. A wound dressing for use in a pressure gradient wound therapy system, the wound dressing comprising:
a wound contact layer having a first surface for contacting a wound and an opposing second surface; and
a covering layer having a first surface facing the wound contact layer and defining a wound dressing cavity, and an opposing second surface;
the covering layer further comprising an aperture configured to provide fluid communication between the wound dressing cavity and a pressure gradient source;
wherein the wound contact layer and the covering later both have an oval shaped outline.

54. A wound dressing according to claim 53 wherein the wound contact layer and covering layer have the same shape.

55. A wound dressing according to claim 53 or 54 wherein the wound contact layer is elliptical.

56. A wound dressing according to any of claims 53 to 55 wherein the covering layer is elliptical.

57. A wound dressing according to any of claims 53 to 56 wherein the covering layer extends an equal distance beyond the perimeter of the wound contact layer about the entire perimeter.

58. A wound dressing according to any of claims 53 to 57 wherein the wound dressing further comprises an absorbent layer and a pressure distribution layer.

59. A wound dressing according to any of claims 53 to 58 wherein the oval shape of the wound contact layer defines a major axis and a minor axis, the major axis and minor axis intersect at a centre point of the wound dressing, and the centre point is the centre of a circular central region of the wound dressing.

60. A wound dressing according to claim 59 wherein the central region has a diameter equal to the width of the wound contact layer.

61. A wound dressing according to claim 59 wherein the central region has a diameter less than the width of the wound contact layer.

62. A wound dressing according to any of claims 59 to 61 wherein the aperture is arranged outside of the central region.

63. A wound dressing according to any of claims 59 to 62 wherein the aperture is arranged on the major axis.

64. A wound dressing according to any of claims 53 to 63 wherein adhesive is provided on the first surface of the covering layer in the region between the wound contact layer perimeter and the covering layer perimeter.

65. A wound dressing according to any of claims 53 to 64 wherein the dressing is a one-piece dressing.

66. A pressure gradient wound therapy system, the system comprising:
a wound dressing comprising a wound contact layer and a covering layer both layers having an oval shaped outline;
a pressure gradient source; and
a first length of pressure tubing.

67. A pressure gradient wound therapy system according to claim 66 wherein the wound dressing is a wound dressing according to any of claims 53 to 65.

68. A pressure gradient wound therapy system according to claim 66 or 67 wherein the system is a negative pressure wound therapy system and the pressure gradient source is a pump.

69. A method of applying a wound dressing comprising a wound contact layer and a covering layer, wherein both layers have an oval shaped outlined to a wound site.

70. A method of applying a wound dressing according to claim 69 wherein the wound dressing is a wound dressing according to any of claims 53 to 65.
